(19) United States
(12) Patent Application Publication
(10) Pub. No.: US 2004/0209370 A1
(43) Pub. Date: Oct. 21, 2004

(54) Method for chromosomal engineering

(76) Inventor: Wonchul Suh, Hockessin, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/734,936

(22) Filed: Dec. 12, 2003

Related U.S. Application Data
(60) Provisional application No. 60/434,602, filed on Dec. 19, 2002.

(51) Int. Cl. C12N 15/74; C12N 1/21

(52) U.S. Cl. 435/488; 435/252.3

(57) ABSTRACT

A method is provided for the integration of foreign genetic elements into a bacterial chromosome without the need of a cloning step. The method relies on the presence of the λ-Red recombinase system in a recombination proficient bacterial host. At least two linear recombination elements or constructs are co-transformed into the recombination proficient host which are assembled in the correction orientation in the bacterial chromosome by homologous recombination. Selectable markers used for the selection of transformants are later excised by the action of a site-specific recombination.

This application claims the benefit of U.S. Provisional Application No. 60/434,602 filed Dec. 19, 2002.

### FIELD OF THE INVENTION

This invention is in the field of microbiology. More specifically, this invention pertains to methods associated with in vivo chromosomal engineering.

### BACKGROUND OF THE INVENTION

The availability of complete bacterial genome sequences and the elucidation of metabolic pathways has resulted in the use of such knowledge to engineer microorganisms for the production of compounds of industrial interest. Microbial production of industrial compounds requires the ability to efficiently engineer changes to the genomes of the organisms. Engineering changes such as adding, removing, or modifying genetic elements has often proven to be challenging and time consuming exercises. One such modification is genetically engineering modulations to the expression of relevant genes in a metabolic pathway.

There are a variety of ways to modulate gene expression. Microbial metabolic engineering generally involves the use of multi-copy vectors to express a gene of interest under the control of a strong or conditional promoter. This method of metabolic engineering for industrial use has several drawbacks. It is sometimes difficult to maintain the vectors due to segregational instability. Deleterious effects on cell viability and growth are often observed due to the vector burden. It is also difficult to control the optimal expression level of desired genes on a vector. To avoid the undesirable effects of using a multi-copy vector, a chromosomal integration approach using homologous recombination via a single insertion of bacteriophage λ, transposons, or other suitable vectors containing the gene of interest has been used. However, this method also has drawbacks such as the need for multiple cloning steps in order to get the gene of interest into a suitable vector prior to recombination. Another drawback is the instability associated with the inserted genes, which can be lost due to excision. Lastly, this method has a limitation associated with multiple insertions and the inability to control the location of the insertion site on a chromosome.

Recently, the use of the λ-Red recombination system for PCR-mediated inactivation of chromosomal genes has been described (Murphy et al., Gene, 246:321-330 (2000); Murphy, K., J. Bacteriol., 180:2063-2071 (1998); Poteete and Fenton, J. Bacteriol., 182:2336-2340 (2000); Poteete, A., FEMS Microbiology Lett., 201:9-14 (2001); Datsenko and Wanner, PNAS, 97:6640-6645 (2000); Yu et al., PNAS, 97:5978-5983 (2000); and Chaveroche et al., Nucleic Acids Research, 28:e97:1-6 (2000)).

Murphy et al. (supra, 1998) illustrate the use of the λ-Red system and how it promotes recombination between bacterial chromosomes and a linear double-stranded (ds) DNA molecule introduced via electroporation. Several strains of bacteria, including Salmonella and enteropathogenic and enterohemorrhagic E. coli were successfully transformed using the system (Poteete, A., supra, 2001). However, the method of Murphy et al. (supra, 1998) used several hundred bases of homologous overlap in order to maximize the recombination efficiency.

Stewart et al. (U.S. Pat. No. 6,355,412) teach the use bacterial recombinases, namely the RecET and/or the λ-Red system, for cloning and subcloning a target DNA fragment into a vector. The target DNA contains termini that share homology to corresponding homology arms on the vector. The target DNA is added to the vector via recombinase mediated homologous recombination. The methods described in U.S. Pat. No. 6,355,412 are limited to adding target DNA to a vector. U.S. Pat. No. 6,355,412 does not teach a two-fragment method of chromosomal engineering nor does it teach a method useful for chromosomally engineering promoter and regulatory regions to artificially control the expression level of desired genes and/or operons in a biosynthetic pathway. A similar approach by Chaveroche et al. (supra) uses the λ-Red system for modification of a cosmid later used to transform via homologous recombination the genome of a filamentous fungi, Asperillus nidulas. Both examples illustrate the use of the λ-Red (or a functionally equivalent) system for genetic modification of a vector later used for homologous recombination with the host's genome or for epichromosomal transformation.

Stewart et al. (U.S. Pat. No. 6,509,156) teach the use of the RecET and/or λ-Red system gene products for catalyzing homologous recombination between two DNA molecules (two regions of homology or “homology arms”). The U.S. Pat. No. 6,509,156 does not teach a method based on triple homologous recombination (recombination between two linear DNA molecules and the bacterial chromosome) nor does it teach a method suitable for chromosomally engineering biosynthetic pathways in host bacteria. The methods described in both Stewart et al. patents would require one or more additional cloning steps prior to homologous recombination in order to integrate an expressible DNA fragment simultaneously with a selectable marker into the host cell's chromosome.

Zhang et al. (Nat Genet, 20:123-128, (1998)) teach the use of the RecET recombinase system in sbcA positive E. coli strains, illustrating the ability to stimulate homologous recombination for cloning and subcloning a target DNA fragment into a vector. Zhang et al. do not provide data on the λ-Red system nor do they teach a method of chromosomal engineering using triple homologous recombination (two DNA fragment method) that make it possible to integrate a promoter or gene along with a selectable marker in a single step without the need of a prior cloning step.

Murphy et al. (supra, 2000) teach the use of the λ-Red system in E. coli and illustrate the utility of the system in creating gene knockouts without the need for a prior cloning step. The λ-Red system was compared to recombination proficient strains of E. coli and was found to be much more efficient, increasing recombination rates approximately 10 to 100-fold. However, the described method required the use of approximately 1000 bp of homology on each end of a single linear dsDNA fragment for efficient transformation. Additionally, the intent of the described method was for open reading frame (ORF) functional analysis by gene knockout, resulting in the identification of unknown essential genes for drug development, but not for chromosomal integration of promoter(s) and/or gene(s).

Pati et al. (U.S. Pat. No. 6,074,853) teach the use of targeted homologous recombination for altering genetic sequences in vivo. Their complex multi-step method relies on the use of two single-stranded polynucleotides coated in vitro with purified E. coli RecA protein. The preferred target for recombination in Pati et al. is an epichromosomal sequence, such as a plasmid or other vector, with the RecA/single-stranded DNA mediated reaction occurring in vitro. The modified vector is then treated to remove the RecA protein prior to introduction into the targeted host cell. The present method is much less complicated and time consuming and targets the host cell's genome directly.

The problem to be solved therefore is to provide methods and materials useful for a facile, one-step method of in vivo chromosomal engineering of promoters and genes in bacteria, such as E. coli. A simple one-step mechanism does not exist which allows for direct modification of chromosomal genes, promoters, and other nucleic acid regulatory elements using homologous recombination.

The present invention provides an easy, one-step, efficient method (lacking a cloning step used in previous methods) to stably integrate any promoter/regulatory region in front of any target gene in the E. coli chromosome so that the expression of such gene can be controlled. Additionally, the present method also permits repetitive cycles of chromosomal modifications for the creation and/or manipulation of biochemical pathways within a host cell using targeted addition and/or deletion of functional DNA molecules via the use triple homologous recombination between two linear, PCR-generated, DNA fragments and the host cell's chromosome.

### SUMMARY OF THE INVENTION

The utility of the present chromosomal integration method in engineering bacterial biosynthetic pathways is illustrated using isoprenoid/carotenoid biosynthesis as an example. The promoters of the key genes that encode for rate-limiting enzymes involved in the isoprenoid pathway are engineered via the present method. The genetic modifications resulted in increased β-carotene production. Genes encoding enzymes involved in carotenoid biosynthesis, not normally found in E. coli, are also added via the present method to specific locations on the host's chromosome under the control of a strong promoter. The method allows for multiple chromosomal modifications within Escherichia coli, which is essential when engineering biosynthetic pathways for industrial purposes. Thus, the present method can be used for multiple chromosomal modifications, useful in engineering biosynthetic pathways. The method of homologous recombination using two linear PCR-generated fragments in the presence of the phage λ-Red recombinase system is described in FIG. 1. The method also includes the use of a site-specific recombinase for removal of selectable markers, allowing easy and efficient in vivo chromosomal engineering.

Accordingly the invention provides a method for the directed integration of an expressible DNA fragment lacking a selectable marker into a bacterial chromosome comprising:

a) providing at least one first recombination element having the general structure in the 5′ to 3′ direction:

5′-RR1-RS-SM-RS-RR2-3′; wherein

(i) RR1 is a first recombination region of about 10 to 50 bases;

(ii) RS is a recombination site responsive to a site-specific recombinase;

(iii) SM is a DNA fragment encoding a selectable marker; and

(iv) RR2 is a second recombination region of about 10 to 50 bases;

b) providing at least one second recombination element having the general structure in a 5′ to 3′ direction:

X-RR3; wherein

(i) X is a DNA is an expressible DNA fragment having homology to the second recombination region; and

(ii) RR3 is a third recombination of about 10-50 bases;

c) providing a recombination proficient bacterial host harboring a λ-Red recombinase system, having a bacterial chromosome comprising:

(i) a first chromosomal region having homology to said first recombination region;

(ii) a second chromosomal region having homology to said third recombination region;

d) transforming said recombination proficient host with the first and second recombination elements, wherein both elements are integrated into the bacterial chromosome between the first and second chromosomal regions forming a construct having the general structure in the 5′ to 3′ direction;

5′-RR1-RS-SM-RS-RR2-X-RR3;

e) selecting and isolating transformed hosts having the construct of (d) on the basis of the selectable marker;

f) expressing a site-specific recombinase in the isolated hosts of (e) wherein the selectable marker is excised from the chromosome and whereby the expressible DNA fragment is inserted into the bacterial chromosome, lacking the selectable marker.

In similar fashion the invention provides a method for the directed integration of an expressible DNA fragment lacking a selectable marker into a bacterial chromosome comprising:

a) providing at least one first recombination element having the general structure in the 5′ to 3′ direction:

5′-RR1-RS-SM-RS-Y-RR2-3′; wherein

(i) RR1 is a first recombination region of about 10 to 50 bases;

(ii) RS is a recombination site responsive to a site-specific recombinase;

(iii) SM is a DNA fragment encoding a selectable marker;

(iv) Y is a first expressible DNA fragment; and

(v) RR2 is a second recombination region of about 10 to 50 bases;

b) providing at least one second recombination element having the general structure in a 5′ to 3′ direction:

5′-X-RR3-3′; wherein

(i) X is a DNA is a second expressible DNA fragment having homology to the second recombination region; and

(ii) RR3 is a third recombination of about 10-50 bases;

c) providing a recombination proficient bacterial host harboring a λ-Red recombinase system, and having a bacterial chromosome comprising:

(i) a first chromosomal region having homology to said first recombination region;

(ii) a second chromosomal region having homology to said third recombination region;

d) transforming said recombination proficient host with the first and second recombination elements, wherein both elements are integrated into the bacterial chromosome between the first and second chromosomal regions forming a construct having the general structure in the 5′ to 3′ direction;

5′-RR1-RS-SM-RS-Y-RR2-X-RR3;

e) selecting and isolating transformed hosts having the construct of (d) on the basis of the selectable marker;

f) expressing a site-specific recombinase in the isolated hosts of (e) wherein the selectable marker is excised from the chromosome and whereby the first and second expressible DNA fragments are inserted into the bacterial chromosome, lacking the selectable marker.

In an alternate embodiment the invention provides a method for the integration of a foreign promoter in place of a bacterial chromosomal promoter in a recombination proficient host cell comprising:

a) providing at least one first recombination element having the general structure in the 5′ to 3′ direction:

5′-RR1-RS-SM-RS-RR2-3′; wherein

(i) RR1 is a first recombination region of about 10 to 50 bases;

(ii) RS is a recombination site responsive to a site-specific recombinase;

(iii) SM is a DNA fragment encoding a selectable marker; and

(iv) RR2 is a second recombination region of about 10 to 50 bases;

b) providing at least one second recombination element having the general structure in a 5′ to 3′ direction:

5′-FP-RR3-3′; wherein

(i) FP is a promoter foreign to the recombination proficient host cell having homology to the second recombination region; and

(ii) RR3 is a third recombination of about 10-50 bases;

c) providing a recombination proficient bacterial host harboring a λ-Red recombinase system, having a bacterial chromosome comprising:

(i) a first chromosomal region upstream of a bacterial promoter having homology to said first recombination region;

(ii) a second chromosomal region, downstream of said bacterial promoter having homology to said third recombination region;

d) transforming said recombination proficient host with the first and second recombination elements, wherein both elements are integrated into the bacterial chromosome between the first and second chromosomal regions forming a construct having the general structure in the 5′ to 3′ direction;

5′-RR1-RS-SM-RS-RR2-FP-RR3;

e) selecting and isolating transformed hosts having the construct of (d) on the basis of the selectable marker;

f) expressing a site-specific recombinase in the isolated hosts of (e) wherein the selectable marker is excised from the chromosome and whereby the foreign promoter is inserted into the bacterial chromosome in place of the bacterial promoter.

In another embodiment the invention provides a method for the integration of an unlinked foreign promoter and foreign open reading frame into a bacterial chromosome in a recombination proficient host cell comprising:

a) providing at least one first recombination element having the general structure in the 5′ to 3′ direction:

5′-RR1-RS-SM-RS-FP-RR2-3′; wherein

(i) RR1 is a first recombination region of about 10 to 50 bases;

(ii) RS is a recombination site responsive to a site-specific recombinase;

(iii) SM is a DNA fragment encoding a selectable marker;

(iv) FP is a promoter foreign to the recombination proficient host cell; and

(iv) RR2 is a second recombination region of about 10 to 50 bases;

b) providing at least one second recombination element having the general structure in a 5′ to 3′ direction:

5′-FO-RR3-3′; wherein

(i) FO is an open reading frame foreign to the recombination proficient host cell having homology to the second recombination region; and

(ii) RR3 is a third recombination of about 10-50 bases;

c) providing a recombination proficient bacterial host harboring a λ-Red recombinase system, having a bacterial chromosome comprising:

(i) a first chromosomal region upstream of a bacterial intra-operon chromosomal integration site having homology to said first recombination region;

(ii) a second chromosomal region, downstream of said bacterial intra-operon chromosomal integration site having homology to said third recombination region;

d) transforming said recombination proficient host with the first and second recombination elements, wherein both elements are integrated into the bacterial chromosome between the first and second chromosomal regions forming a construct having the general structure in the 5′ to 3′ direction;

5′-RR1-RS-SM-RS-FP-RR2-FO-RR3;

e) selecting and isolating transformed hosts having the construct of (d) on the basis of the selectable marker;

f) expressing a site-specific recombinase in the isolated hosts of (e) wherein the selectable marker is excised from the chromosome and whereby the foreign promoter and foreign open reading frame are inserted into the bacterial chromosome.

In another preferred embodiment the invention provides a method for the integration of a foreign gene comprising a regulatory region and foreign open reading frame into a bacterial chromosome in a recombination proficient host cell comprising:

a) providing at least one first recombination element having the general structure in the 5′ to 3′ direction:

5′-RR1-RS-SM-RS-FG-RR2-3′; wherein

(i) RR1 is a first recombination region of about 10 to 50 bases;

(ii) RS is a recombination site responsive to a site-specific recombinase;

(iii) SM is a DNA fragment encoding a selectable marker;

(iv) FG is a gene comprising a regulatory region, foreign to the recombination proficient host cell; and

(iv) RR2 is a second recombination region of about 10 to 50 bases;

b) providing at least one second recombination element having the general structure in a 5′ to 3′ direction:

5′-FO-RR3-3′; wherein

(i) FO is an open reading-frame foreign to the recombination proficient host cell having homology to the second recombination region; and

(ii) RR3 is a third recombination of about 10-50 bases;

c) providing a recombination proficient bacterial host harboring a λ-Red recombinase system, having a bacterial chromosome comprising:

(i) a first chromosomal region upstream of a bacterial intra-operon chromosomal integration site having homology to said first recombination region;

(ii) a second chromosomal region, downstream of said bacterial intra-operon chromosomal integration site having homology to said third recombination region;

d) transforming said recombination proficient host with the first and second recombination elements, wherein both elements are integrated into the bacterial chromosome between the first and second chromosomal regions forming a construct having the general structure in the 5′ to 3′ direction;

5′-RR1-RS-SM-RS-FG-RR2-FO-RR3;

e) selecting and isolating transformed hosts having the construct of (d) on the basis of the selectable marker;

f expressing a site-specific recombinase in the isolated hosts of (e) wherein the selectable marker is excised from the chromosome and whereby the foreign promoter and foreign open reading frame are inserted into the bacterial chromosome.

### BRIEF DESCRIPTION OF THE DRAWINGS, SEQUENCE DESCRIPTIONS AND BIOLOGICAL DEPOSITS

FIG. 1 illustrates the general method used in the present invention for a variety of in vivo bacterial chromosomal modifications.

FIG. 2 illustrates the method of chromosomal promoter engineering of the dxs gene using kanamycin selectable marker and phage T5 promoter (P_{T5})

FIG. 3 illustrates the isoprenoid/carotenoid biosynthetic pathway.

FIG. 4 illustrates the λ-Red helper plasmid, pKD46 (Datsenko and Wanner, supra; GenBank® Accession Number AY048746).

FIG. 5 illustrates the pPCB15 reporter plasmid construct containing several genes involved in carotenoid biosynthesis.

FIG. 6 illustrates the engineered chromosomal promoter construct and the PCR fragment analysis illustrating the kan-P_{T5}-dxs and P_{T5}-dxs constructs.

FIG. 7 illustrates the effects on β-carotene production by the introduction of a phage T5 promoter (P_{T5}) upstream of several isoprenoid genes.

FIG. 8 illustrates the method for introduction of the P_{T5} promoter upstream of the idi gene and the removal of the antibiotic selection marker.

FIG. 9 illustrates the PCR fragment analysis illustrating the kan-P_{T5}-idi and P_{T5}-idi constructs.

FIG. 10 is a plasmid map of pSUH5 plasmid used for the preparation of the fused antibiotic marker-P_{T5} promoter(kan-P_{T5}) PCR-generated DNA fragment.

FIG. 11 illustrates the method for the introduction of the fused antibiotic marker kan-P_{T5} promoter and the Methylomonas sp. 16a dxs gene into the E. coli chromosome and the removal of the antibiotic selection marker.

FIG. 12 illustrates the PCR fragment analysis illustrating the kan-P_{T5}-dxs(16a) and P_{T5}-dxs(16a) constructs.

FIG. 13 illustrates the method for the introduction of the fused antibiotic marker kan-P_{T5} promoter and P. stewartii crtEIB genes into the E. coli chromosome.

FIG. 14 illustrates the PCR fragment analysis illustrating the kan-P_{T5}-crtEIB and P_{T5}-crtEIB constructs.

The invention can be more fully understood from the following detailed description and the accompanying sequence descriptions, which form a part of this application.

The following sequences comply with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

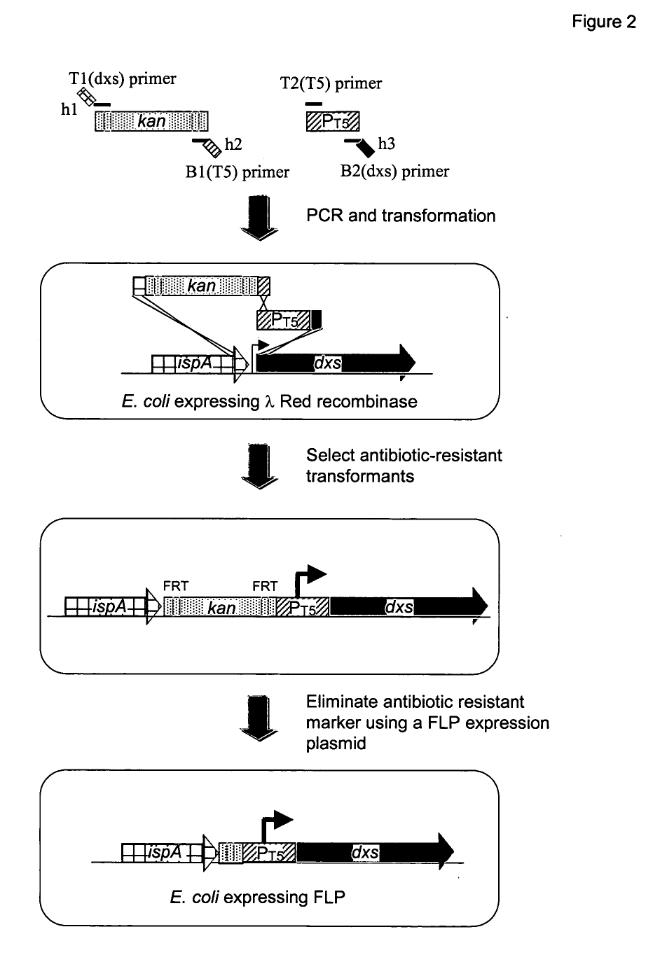

SEQ ID NO:1 is the first of two primer sequences used to PCR amplify the linear DNA fragment containing a kanamycin resistance gene from plasmid pKD4 (Datsenko and Wanner., supra.; GenBank® Accession Number AY048743) designated as “T1(dxs)” and contains a homology arm designated as “h1” chosen to match sequences in the upstream region of the ispA stop codon (FIG. 2).

SEQ ID NO:2 is the second of two primer sequences used to PCR amplify the linear DNA fragment containing a kanamycin resistance gene from plasmid pKD4 designated as “B1(T5)” and contains a homology arm designated as “h2” chosen to match sequences in the 5′-end region of the phage T5 promoter (P_{T5}) DNA fragment (FIG. 2).

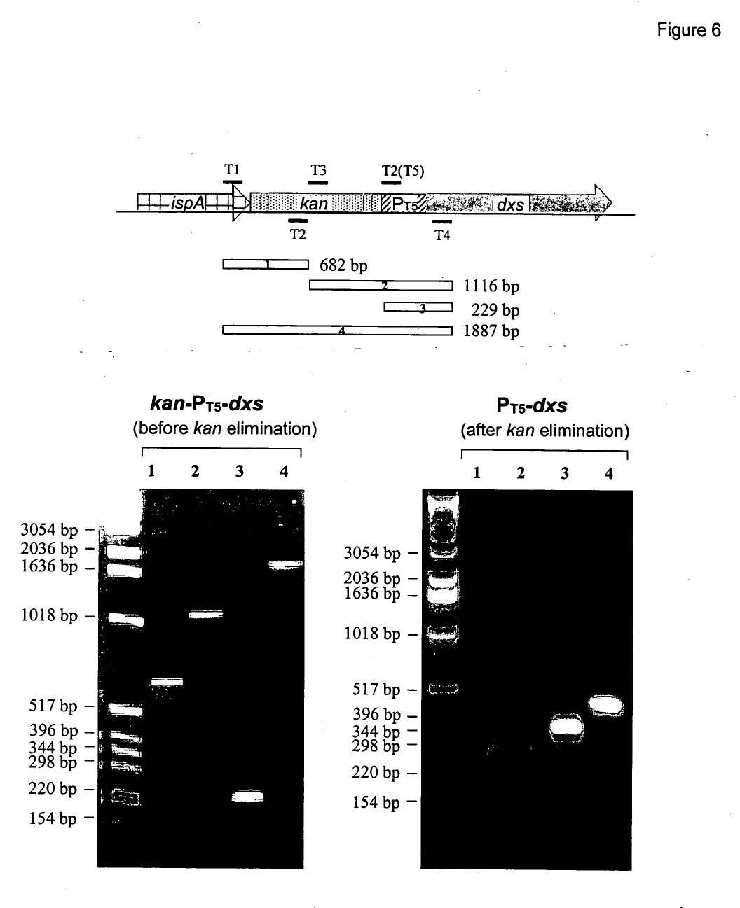

SEQ ID NO:3 is the first of two primer sequence used to PCR amplify the linear DNA fragment containing a P_{T5} promoter comprising the −10 and −35 consensus sequences, lac operator (lacO), and ribosomal binding site (rbs) from plasmid pQE30 (Qiagen Inc., Valencia, Calif.) and is designated as “T2(T5)” (FIG. 2). The primer is later used to verify by PCR amplification the chromosomal construct of the integration of the kanamycin selectable marker and the P_{T5} promoter upstream of the dxs gene (FIG. 6).

SEQ ID NO:4 is the second of two primer sequences used to PCR amplify the linear DNA fragment containing a P_{T5} promoter comprising the −10 and −35 consensus sequences, lac operator (lacO), and ribosomal binding site (rbs) from pQE30 (Qiagen, Inc.) and is designated as “B2(dxs)” and contains a homology arm designated as “h3” chosen to match sequences in the downstream region of the dxs start codon (FIG. 2).

SEQ ID NO:5 is a primer used to verify by PCR amplification the chromosomal construct of the integration of the kanamycin selectable marker and the P_{T5} promoter upstream of the dxs gene and is designated as “T1” (FIG. 6).

SEQ ID NO:6 is a primer used to verify by PCR amplification the chromosomal construct of the integration of the kanamycin selectable marker and the P_{T5} promoter upstream of the dxs gene and is designated as “T2” (FIG. 6).

SEQ ID NO:7 is a primer used to verify by PCR amplification the chromosomal construct of the integration of the kanamycin selectable marker and the P_{T5} promoter upstream of the dxs gene and is designated as “T3” (FIG. 6).

SEQ ID NO:8 is a primer used to verify by PCR amplification the chromosomal construct of the integration of the kanamycin selectable marker and the P_{T5} promoter upstream of the dxs gene and is designated as “T4” (FIG. 6).

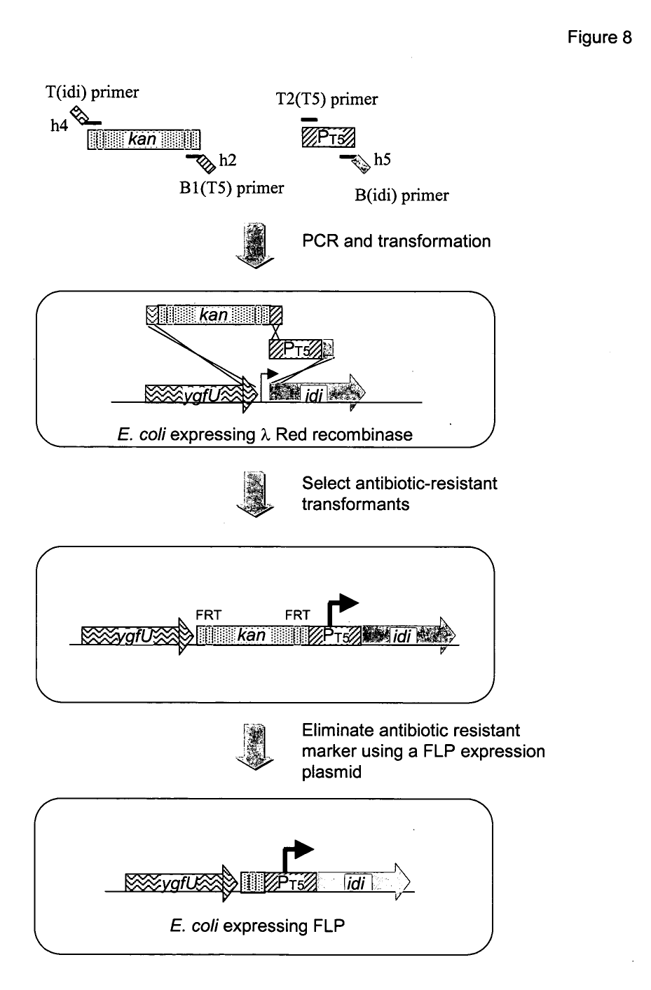

SEQ ID NO:9 is the first of a primer pair used to PCR amplify a DNA fragment containing a kanamycin resistance selectable marker from plasmid pKD4 and is designated as “T(idi)” and contains a homology arm designated as “h4” chosen to match sequences in the upstream region of the ygfU stop codon (FIG. 8).

SEQ ID NO:10 is the second of a primer pair used to PCR amplify a DNA fragment containing a P_{T5} promoter comprising the −10 and −35 consensus sequences, lac operator (lacO), and ribosomal binding site (rbs) from pQE30 (Qiagen, Inc.) and is designated as “B(idi)” and contains a homology arm designated as “h5” chosen to match sequences in the downstream region of the idi start codon (FIG. 8).

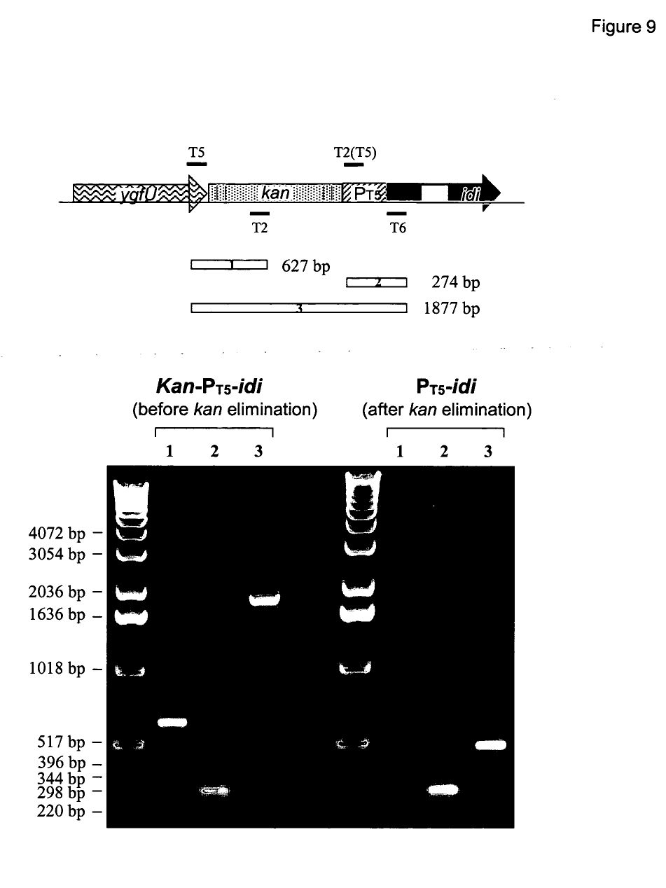

SEQ ID NO:11 is a primer used for PCR analysis confirming chromosomal integration of the kanamycin selectable marker and the P_{T5} promoter upstream of the idi gene and is designated as “T5” (FIG. 9).

SEQ ID NO:12 is a primer used for PCR analysis confirming chromosomal integration of the kanamycin selectable marker and the P_{T5} promoter upstream of the idi gene and is designated as “T6” (FIG. 9).

SEQ ID NO:13 is the first of a primer pair used to PCR amplify a DNA fragment containing the P_{T5} promoter element comprising the −10 and −35 consensus sequences, lacO, and ribosomal binding site (rbs) from pQE30 (Qiagen, Inc.) and is designated as “Tt5”.

SEQ ID NO:14 is the second of a primer pair used to PCR amplify a DNA fragment containing the P_{T5} promoter element comprising the −10 and −35 consensus sequences, lacO, and ribosomal binding site (rbs) from pQE30 (Qiagen, Inc.) and is designated as “Bt5”.

SEQ ID NO:15 is the nucleotide sequence of the ORF encoding the D-1-deoxyxylulose-5-phosphate synthase (DXS) enzyme from Methylomonas 16a which catalyzes the condensation of pyruvate and D-glyceraldehyde 3-phosphate to D-1-deoxyxylulose 5-phosphate.

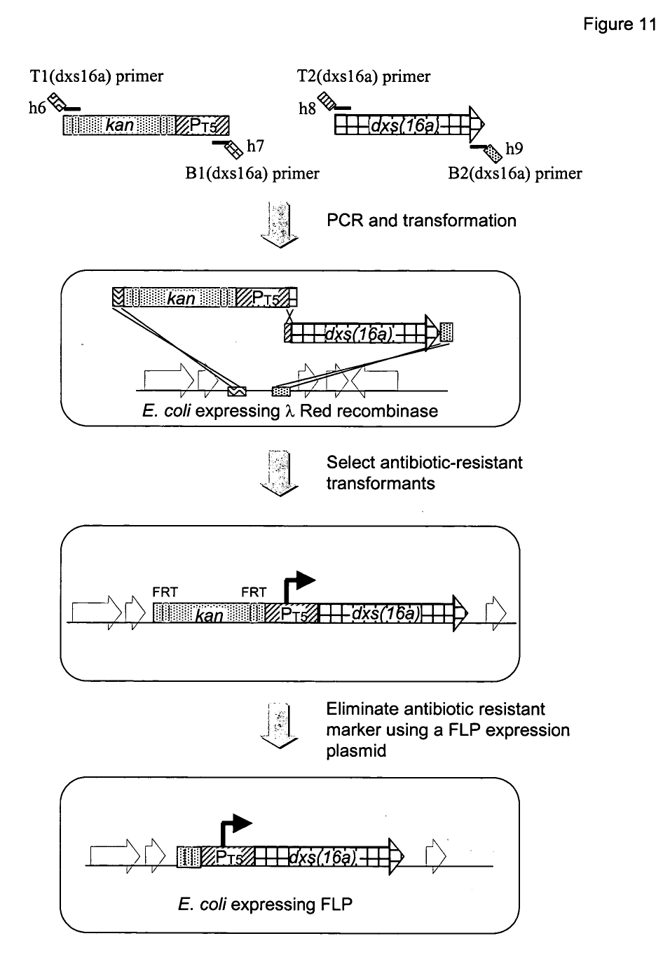

SEQ ID NO:16 is the first of a primer pair used to PCR amplify a DNA fragment containing a fused kanamycin selectable marker-P_{T5} promoter from pSUH5 and is designated as “T1(dxs16a)” and contains a homology arm designated as “h6” chosen to match sequence in the region between genes located at 30.9 min of the E. coli chromosome (FIG. 11).

SEQ ID NO:17 is the second of a primer pair used to PCR amplify a DNA fragment containing a fused kanamycin selectable marker-P_{T5} promoter from pSUH5 and is designated as “B1(dxs16a)” and contains a homology arm designated as “h7” chosen to match sequence in the downstream region of the dxs(16a) start codon (FIG. 11).

SEQ ID NO:18 is the first of a primer pair used to PCR amplify a DNA fragment containing Methylomonas dxs(16a) gene from Methylomonas 16a genomic DNA and is designated as “T2(dxs16a)” and contains a homology arm designated as “h8” chosen to match sequences in the 3′-end region of the fused kanamycin selectable marker-P_{T5} promoter (FIG. 11).

SEQ ID NO:19 is the second of a primer pair used to PCR amplify a DNA fragment containing Methylomonas dxs(16a) gene from Methylomonas 16a genomic DNA and is designated as “B2(dxs16a)” and contains a homology arm designated as “h9” chosen to match sequences in the region between genes located at 30.9 minutes of the E. coli chromosome (FIG. 11).

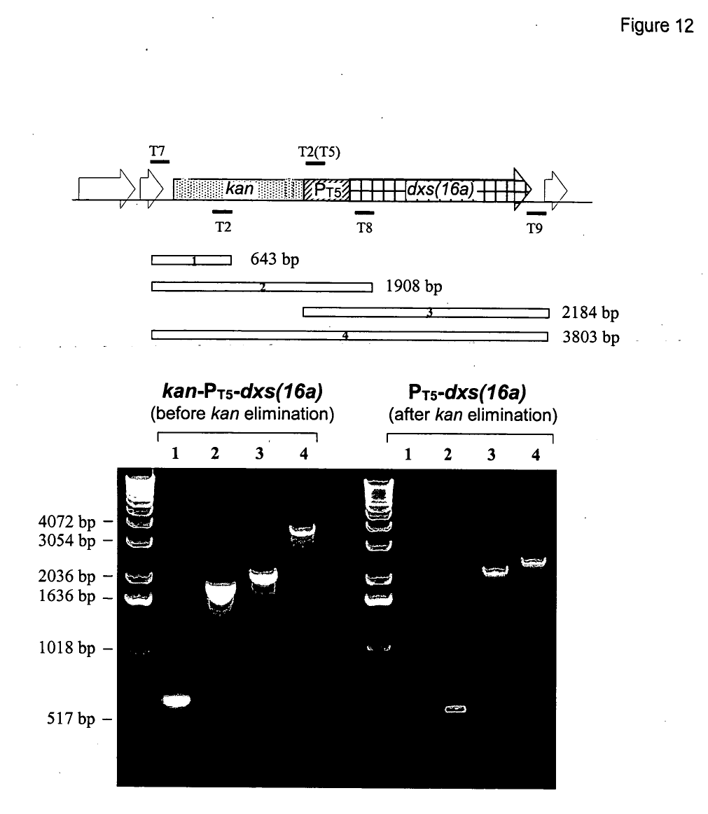

SEQ ID NO:20 is a primer used for PCR analysis confirming chromosomal integration of the fused kan-P_{T5} promoter and dxs(16a) gene and is designated as “T7” (FIG. 12).

SEQ ID NO:21 is a primer used for PCR analysis confirming chromosomal integration of the fused kan-P_{T5} promoter and dxs(16a) gene and is designated as “T8” (FIG. 12).

SEQ ID NO:22 is a primer used for PCR analysis confirming chromosomal integration of the fused kan-P_{T5} promoter and dxs(16a) gene and is designated as “T9” (FIG. 12).

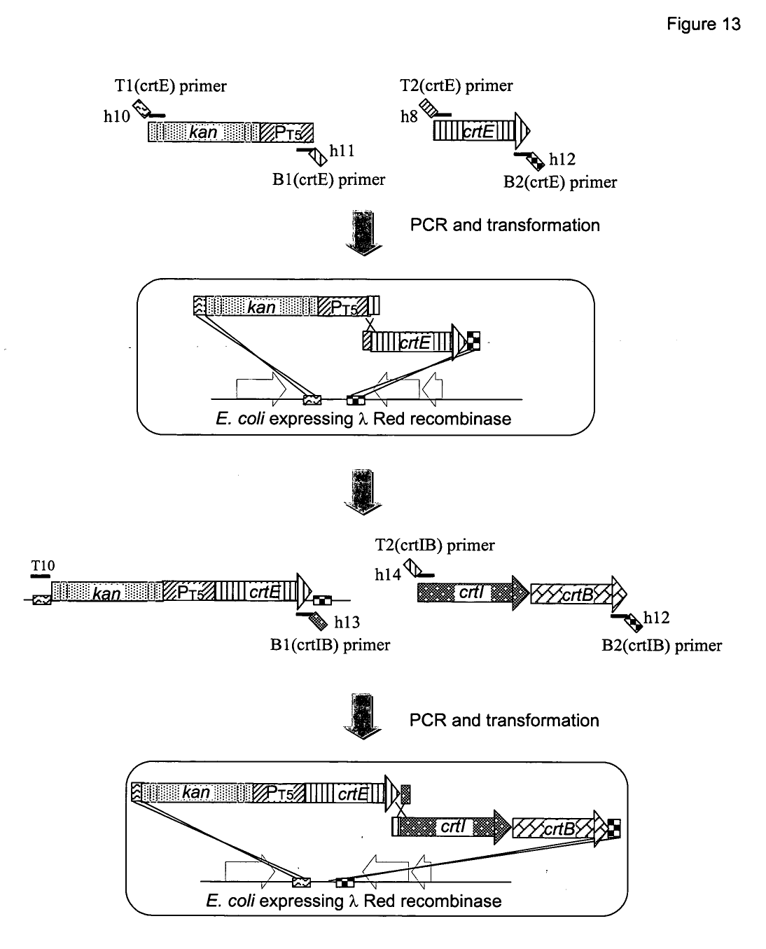

SEQ ID NO:23 is the first of a primer pair used to PCR amplify a DNA fragment containing a fused kanamycin selectable marker-P_{T5} promoter from pSUH5 and is designated as “T1 (crtE)” and contains a homology arm designated as “h10” chosen to match sequence in the inter-operon region located at 81.2 minutes of the E. coli chromosome (FIG. 13).

SEQ ID NO:24 is the second of a primer pair used to PCR amplify a DNA fragment containing a fused kanamycin selectable marker-P_{T5} promoter from pSUH5 and is designated as “B1(crtE)” and contains a homology arm designated as “h11” chosen to match sequence in the downstream region of the crtE start codon (FIG. 13).

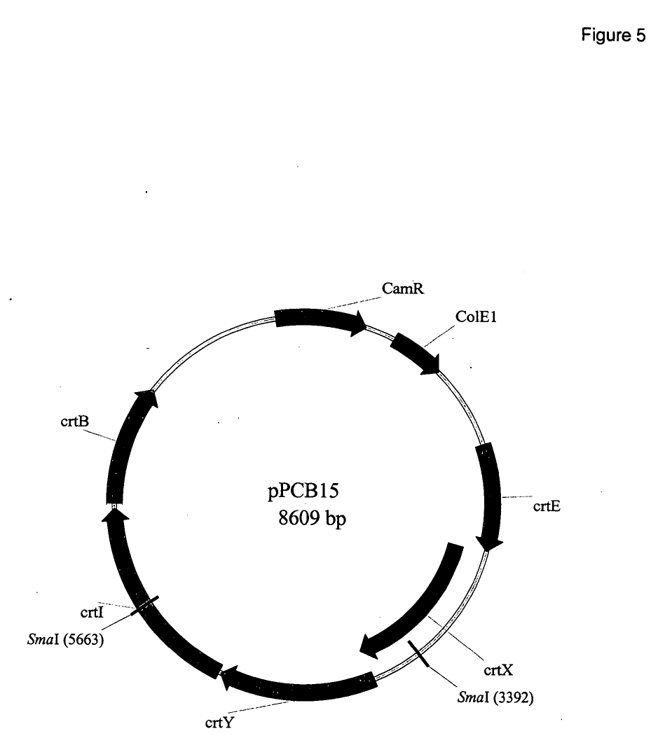

SEQ ID NO:25 is the first of a primer pair used to PCR amplify a DNA fragment containing P. stewartii crtE gene from pPCB15 (FIG. 5) and is designated at “T2(crtE)” and contains a homology arm designated as “h8” chosen to match sequences in the 3′-end region of the fused kanamycin selectable marker-P_{T5} promoter (FIG. 13).

SEQ ID NO:26 is the second of a primer pair used to PCR amplify a DNA fragment containing P. stewartii crtE gene from pPCB15 and is designated at “B2(crtE)” and contains a homology arm designated “h12” chosen to match sequences in the inter-operon region located at 81.2 minutes on the E. coli chromosome (FIG. 13).

SEQ ID NO:27 is a primer used for PCR amplify a DNA fragment containing a priming sequence (26 bp) corresponding to the 162 bases upstream region of the integration site of the fused kanamycin marker-P_{T5} promoter-P. stewartii crtE gene from E. coli P_{T5}-crtE and is designated as “T10” (FIG. 13).

SEQ ID NO:28 is the second of a primer pair used to amplify the fused kanamycin marker-P_{T5} promoter-P. stewartii crtE gene from E. coli P_{T5}-crtE and is designated as “B1(crtIB)” and contains a homology arm designated as “h13” chosen to match sequences in the downstream region of the crtI start codon (FIG. 13).

SEQ ID NO:29 is the first of a primer pair used to PCR amplify the P. stewartii crtIB linked gene pair from the plasmid pPCB15 and is designated as “T2(crtIB)” and contains a homology arm designated as “h14” chosen to match sequences in the 3′-end region of the fused kanamycin selectable marker-P_{T5} promoter-P. stewartii crtE DNA fragment (FIG. 13).

SEQ ID NO:30 is the second of a primer pair used to PCR amplify the P. stewartii crtIB linked gene pair from the plasmid pPCB15 and is designated as “B2(crtIB)” and contains a homology arm designated “h12” (same arm used for “B2(crtE)” primer) chosen to match sequences in the inter-operon region located at 81.2 min on the E. coli chromosome (FIG. 13).

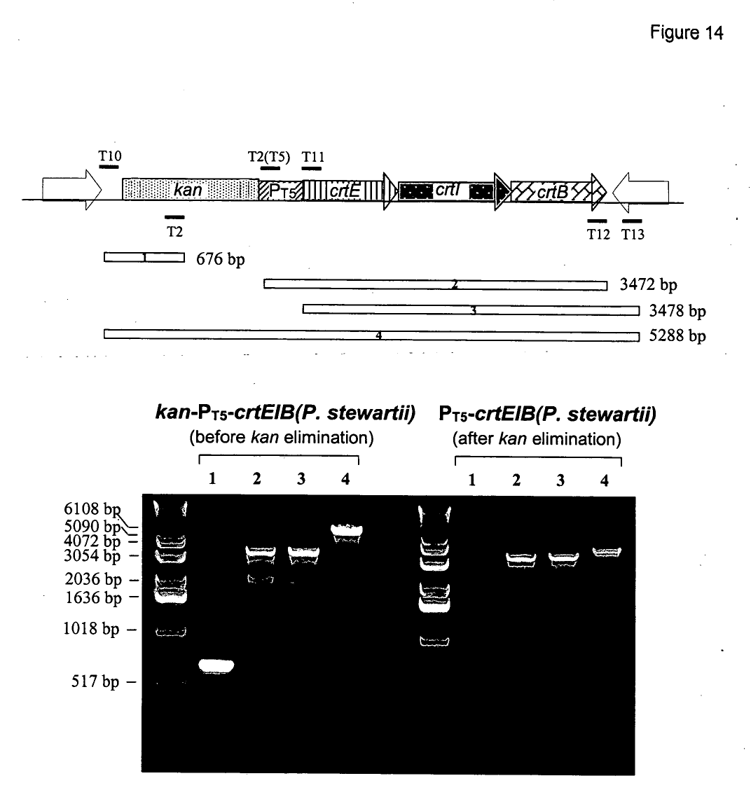

SEQ ID NO:31 is a primer used to verify by PCR amplification the chromosomal construct of the integration of the fused kanamycin selectable marker-P_{T5} promoter-P. stewartii crtE and the P. stewartii crtIB operon in the inter-operon region located at 81.2 min on the E. coli chromosome and is designated as “T11” (FIG. 14).

SEQ ID NO:32 is a primer used to verify by PCR amplification the chromosomal construct of the integration of the fused kanamycin selectable marker-P_{T5} promoter-P. stewartii crtE and the P. stewartii crtIB operon in the inter-operon region located at 81.2 min on the E. coli chromosome and is designated as “T12” (FIG. 14).

SEQ ID NO:33 is a primer used to verify by PCR amplification the chromosomal construct of the integration of the fused kanamycin selectable marker-P_{T5} promoter-P. stewartii crtE and the P. stewartii crtIB operon in the inter-operon region located at 81.2 min on the E. coli chromosome and is designated as “T13” (FIG. 14).

SEQ ID NO:34 is the nucleotide sequence for plasmid pKD4 (Datsenko and Wanner, supra) having GenBank® Accession number AY048743 and was used as a PCR template to amplify the DNA fragment containing a kanamycin resistance gene flanked by FRT site-specific recombinase recognition sequences.

SEQ ID NO:35 is the nucleotide sequence for plasmid pKD46 (Datsenko and Wanner, supra) having GenBank® Accession number AY048746. Plasmid pKD46 expresses the components of the λ-Red Recombinase system.

SEQ ID NO:36 is the nucleotide sequence for plasmid pSUH5 which was used as a template for PCR amplifying the fused kanamycin resistance gene-P_{T5} promoter.

SEQ ID NO:37 is the nucleotide sequence for plasmid pPCB15 which was used as a reporter plasmid construct containing several genes involved in carotenoid biosynthesis, and as a template for PCR amplifying the crtE, and crtIB genes.

Applicants have made the following biological deposit under the terms of the Budapest Treaty on the International Recognition of the Deposit of Microorganisms for the purposes of Patent Procedure:

| Depositor Identification | Int'l. Depository | |
|---|---|---|
| Reference | Designation | Date of Deposit |
| Plasmid pCP20 | ATCC PTA-4455 | Jun. 13, 2002 |
| Methylomonas 16a | ATCC PTA-2402 | Aug. 22, 2000 |

As used herein, “ATCC” refers to the American Type Culture Collection International Depository Authority located at ATCC, 10801 University Blvd., Manassas, Va. 20110-2209, USA. The “International Depository Designation” is the accession number to the culture on deposit with ATCC.

The listed deposits will be maintained in the indicated international depository for at least thirty (30) years and will be made available to the public upon the grant of a patent disclosing it. The availability of a deposit does not constitute a license to practice the subject invention in derogation of patent rights granted by government action.

### DETAILED DESCRIPTION OF THE INVENTION

The invention relates to a method for the directed integration of promoters and/or expressible DNA fragments into a bacterial chromosome in a single step. The method employs at least two linear double-stranded (ds) recombination elements or constructs that carry various expressible DNA elements such as promoters, foreign genes or open reading frames to be chromosomally integrated. Each recombination element comprises a recombination region having regions of homology to either the other recombination element or portions of the bacterial chromosome. A unique feature of the present method is the use of at least two linear double-stranded (ds) recombination elements to chromosomally integrate various expressible DNA elements such as a promoter or foreign gene or open reading frame on one recombination element in combination with a selectable marker on the other recombination element which is bounded by site-specific recombinase sites to allow for facile excision when the marker is no longer needed. Another feature of the present method is the use of the λ-Red recombinase system in a recombination proficient host. The λ-Red system increases the utility of the method by facilitating efficient recombination between linear recombination elements and the chromosome over relatively small regions of homology. The present method facilitates the replacement of native bacterial chromosomal promoters with foreign or artificial promoters. Promoter additions and/or replacements can be combined with chromosomal integration of foreign genes for tunable expression by placing the foreign genes under the control of a regulatory promoter.

The present method can be used to perform additional chromosomal modifications other than promoter replacement, including the chromosomal integration of one or more foreign genes. Functional expression of these modifications was measured by an increase in the production of β-carotene (orange color) or by the production of lycopene (pink color).

In this disclosure, a number of terms and abbreviations are used. The following definitions are provided.

“Open reading frame” is abbreviated ORF.

“Polymerase chain reaction” is abbreviated PCR.

The term “directed integration” or “targeted integration” means the integration of an expressible DNA fragment into a bacterial chromosome whereby the integration is effected by corresponding regions of homology on the a cassette comprising the expressible DNA and the chromosome.

The term “expressible DNA fragment” means any DNA that influences phenotypic changes in the host cell. An “expressible DNA fragment” may include for example, DNA comprising regulatory elements, isolated promoters, open reading frames, genes, or combinations thereof.

The term “recombination element” refers to a linear nucleic acid construct useful for the transformation of a recombination proficient bacterial host. Recombination elements of-the invention may include a variety of genetic elements such as selectable markers, expressible DNA fragments, and recombination regions having homology to regions on a bacterial chromosome or on other recombination elements.

The terms “recombination region” and “homology arm” are used interchangeably and refer to a nucleotide sequence that enables homologous recombination between two nucleic acids having substantially the same nucleotide sequence in a particular region of two different nucleic acids. The preferred size range of the nucleotide sequence of the homology arm is from about 10 to about 50 nucleotides.

The term “recombinase” refers to one or more enzymes, which either work alone or in combination to stimulate homologous recombination. The “λ-Red recombinase”, “λ-Red recombination system”, and “λ-Red system” are used interchangeably to describe a group of enzymes encoded by the bacteriophage λ genes exo, bet, and gam. The enzymes encoded by the three genes work together to increase the rate of homologous recombination in E. coli, an organism generally considered to have a relatively low rate of homologous recombination; especially when using linear recombination elements.

The term “site-specific recombinase system” is used to describe a system comprised of one or more enzymes which recognize specific nucleotide sequences (recombination target sites) and which catalyze recombination between the recombination target sites. Site-specific recombination provides a method to rearrange, delete, or introduce exogenous DNA. Examples of site-specific recombinases and their associated recombination target sites are flippase (FLP)/FRT, Cre/lox, Xer/dif, and Int/att, to name a few The present invention illustrates the use of a site-specific recombinase to remove selectable markers. Antibiotic resistance markers, flanked on both sides by FRT recombination target sites, are removed by expression of the FLP site-specific recombinase. The use of site-specific recombinases to remove selectable markers permits multiple chromosomal modifications necessary for microbial pathway engineering and is not limited to the number of available selection markers (Huang et al., J. Bacteriol., 179(19): 6076-6083 (1997)).

The term “selectable marker” means a gene product that, when present, enables one to identify and preferentially propagate a particular cell type.

The term “recombination proficient bacterial host” is used to describe a bacterial host that contains a functional recombination system and is capable of homologous recombination at rates useful for genetic engineering.

The term “homology” as applied to recombination regions and corresponding regions on a bacterial chromosome means nucleotide sequences sharing identical or nearly identical sequences. Complementary sequences between regions on the bacterial chromosome and recombination regions can associate and undergo homologous recombination in the presence of a recombinase system. Preferred recombination regions, or homology arms, are those having identical sequences to the corresponding regions on the bacterial chromosome and are about 10 to about 50 bp in length.

As used herein the term “upstream” when used in reference to a region of DNA means the 5′ side of a particular gene or sequence of nucleotides.

As used herein the term “downstream” when used in reference to a region of DNA means the 3′ side of a particular gene or sequence of nucleotides.

The term “inter-operon chromosomal integration site” refers to a chromosomal site where integration of exogenous DNA using the current invention is targeted and where integration of the exogenous DNA will not disrupt the functionality of an endogenous operon or gene within the host.

The term “regulatory circuit” refers to functionally integrated DNA which can interact with a substance (signaling molecule) to either increase or decrease transcription. For example, the material could be a repressor protein that binds to an operator site (specific DNA sequence located near or overlapping a promoter's RNA polymerase binding site). Binding of the repressor hinders or prevents transcription of the associated gene. Conversely, the substance may be an activator protein that binds to a specific DNA sequence which facilitates RNA polymerase binding, “activating” or increasing transcription of an associated gene. In the present invention, the term “positive regulatory site” refers to functionally integrated DNA that (either alone or in response to interaction with a substance) enhances transcription of an associated coding sequence. The term “negative regulatory site” refers to functionally integrated DNA that (either alone or in response to interaction with a substance) decreases or blocks transcription of an associated coding sequence.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

The term “isoprenoid” or “terpenoid” refers to the compounds and any molecules derived from the isoprenoid pathway including 10 carbon terpenoids and their derivatives, such as carotenoids and xanthophylls.

The terms “Methylomonas 16a strain” and “Methylomonas 16a” (ATCC PTA-2402, deposited Aug. 22, 2000) are used interchangeably and refer to a bacterium of a physiological group of bacteria known as methanotrophs, which are unique in their ability to utilize methane as a substrate.

The term “methanotroph” means a prokaryote capable of utilizing methane as a substrate. Complete oxidation of methane to carbon dioxide occurs by aerobic degradation pathways. Typical examples of methanotrophs include, but are not limited to the genera Methylomonas, Methylobacter, Methylococcus, and Methylosinus.

The term “dxs” refers to the enzyme D-1-deoxyxylulose 5-phosphate encoded by the E. coli dxs gene which catalyzes the condensation of pyruvate and D-glyceraldehyde 3-phosphate to D-1-deoxyxylulose 5-phosphate.

The term “dxs(16a)” refers to the enzyme D-1-deoxyxylulose 5-phosphate encoded by the Methylomonas 16a strain dxs gene which catalyzes the condensation of pyruvate and D-glyceraldehyde 3-phosphate to D-1-deoxyxylulose 5-phosphate.

The term “idi” refers to the enzyme isopentenyl diphosphate isomerase encoded by the E. coli idi gene that converts isopentenyl diphosphate to dimethylallyl diphosphate.

The term “triple homologous recombination” in the present invention refers to a genetic recombination between two linear DNA fragments and the target chromosome via their homologous sequences resulting in chromosomal integration of the two linear nucleic acid fragments into the target chromosome.

The term “pPCB15” refers to the plasmid containing β-carotene synthesis genes Pantoea crtEXYIB, used as a reporter plasmid for monitoring β-carotene production in E. coli that is genetically engineered via the present method.

The term “pSUH5” refers to the plasmid that was constructed by cloning a phage T5 promoter (P_{T5}) region into the Ndel restriction endonuclease site of pKD4 (Datsenko and Wanner, supra). It was used as a template plasmid for PCR amplification of a fused kanamycin selectable marker-P_{T5} promoter linear DNA molecule.

The terms “P_{T5} promoter” and “phage T5 promoter” refer to the nucleotide sequence that comprises the −10 and −35 consensus sequences from phage T5, lactose operator (lacO), and ribosomal binding site (rbs) from pQE30 (Qiagen, Inc.).

The term “helper plasmid” refers to either pKD46 encoding λ-Red recombinase system or pCP20 encoding the FLP site-specific recombinase (Datsenko and Wanner, supra).

The term “E. coli” when applied to Escherichia coli K-12 derivatives will mean the MG1655 (ATCC 47076) and MC1061 (ATCC 53338) strains

The term “Pantoea stewartii subsp. stewartii” is abbreviated as “Pantoea stewartii” (ATCC 8199) and is used interchangeably with Erwinia stewartii (Mergaert et al., Int J. Syst Bacteriol., 43:162-173 (1993)).

The term “Pantoea crtEXYIB cluster” refers to a gene cluster containing carotenoid synthesis genes crtEXYIB amplified from Pantoea stewartii ATCC 8199. The gene cluster contains the genes crtE, crtX, crtY, crtI, and crtB. The cluster also contains a crtZ gene organized in opposite direction adjacent to crtB gene. In the present invention, the gene cluster is located on the reporter plasmid pPCB15 and is used to monitor the effects of various chromosomal modifications on β-carotene production.

The term “CrtE” refers to geranylgeranyl pyrophosphate synthase enzyme encoded by crtE gene which converts trans-trans-farnesyl diphosphate+isopentenyl diphosphate to pyrophosphate+geranylgeranyl diphosphate.

The term “CrtY” refers to lycopene cyclase enzyme encoded by crtY gene which converts lycopene to beta-carotene.

The term “CrtI” refers to phytoene dehydrogenase enzyme encoded by crtI gene which converts phytoene into lycopene via the intermediaries of phytofluene, zeta-carotene and neurosporene by the introduction of 4 double bonds.

The term “CrtB” refers to phytoene synthase enzyme encoded by crtB gene which catalyzes reaction from prephytoene diphosphate (geranylgeranyl pyrophosphate) to phytoene.

The term “CrtX” refers to zeaxanthin glucosyl transferase enzyme encoded by crtX gene which converts zeaxanthin to zeaxanthin-β-diglucoside.

The term “CrtZ” refers to the β-carotene hydroxylase enzyme encoded by crtZ gene which catalyses both the hydroxylation reaction from β-carotene to zeaxanthin or the hydroxylation of canthaxanthin to astaxanthin.

The term “carotenoid biosynthetic enzyme” is an inclusive term referring to any and all of the enzymes encoded by the crtEXYIB gene cluster. The enzymes include CrtE, CrtY, CrtI, CrtB, and CrtX.

“Codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign gene” or “exogenous gene” refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Operon”, in bacterial DNA, is a cluster of contiguous genes transcribed from one promoter that gives rise to a polycistronic mRNA.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structures.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity. As used herein a “foreign promoter” will refer to a promoter derived from a source other than the cell in which the promoter is being used. For the purposes of the present invention foreign promoters used in the present microbial systems may be derived from other microbes, such as bacteria, yeasts and fungi, as well as eukaryotic sources such as plants and mammalian cells.

“RNA transcript” refers to the product resulting from RNA polymerase-catalyzed transcription of a DNA sequence. When the RNA transcript is a perfect complementary copy of the DNA sequence, it is referred to as the primary transcript or it may be an RNA sequence derived from post-transcriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to an RNA transcript that is complementary to all or part of a target primary transcript or mRNA and that blocks the expression of a target gene (U.S. Pat. No. 5,107,065; WO 9928508). The complementarity of an antisense RNA may be with any part of the specific gene transcript, i.e., at the 5′ non-coding sequence, 3′ non-coding sequence, or the coding sequence. “Functional RNA” refers to antisense RNA, ribozyme RNA, or other RNA that is not translated yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The terms “plasmid”, “vector” and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host.

The invention relates to a method for the directed integration of expressible DNA into a bacterial chromosomal. The DNA is directed to the bacterial chromosome by homologous recombination between regions of homology on a cassette comprising the DNA and the chromosome in a recombination proficient bacterial host harboring the λ-Red recombinase system. At lest two linear cassettes or recombination elements are employed. Typically one recombination element comprises a selectable marker bounded by site-specific recombinase sites. Both 5′ and 3′ to the recombinase sites are recombination regions (“homology arms”) having homology to either portions of the bacterial chromosome or to another recombination element. Additionally the recombination element may also comprise expressible DNA such as a promoter or foreign gene or open reading frame. The second recombination element will typically contain many of the same elements as the first, with the exception of the selectable marker bounded by recombination target sites (recognized by a site-specific recombinase).

λ-Red Recombinase System

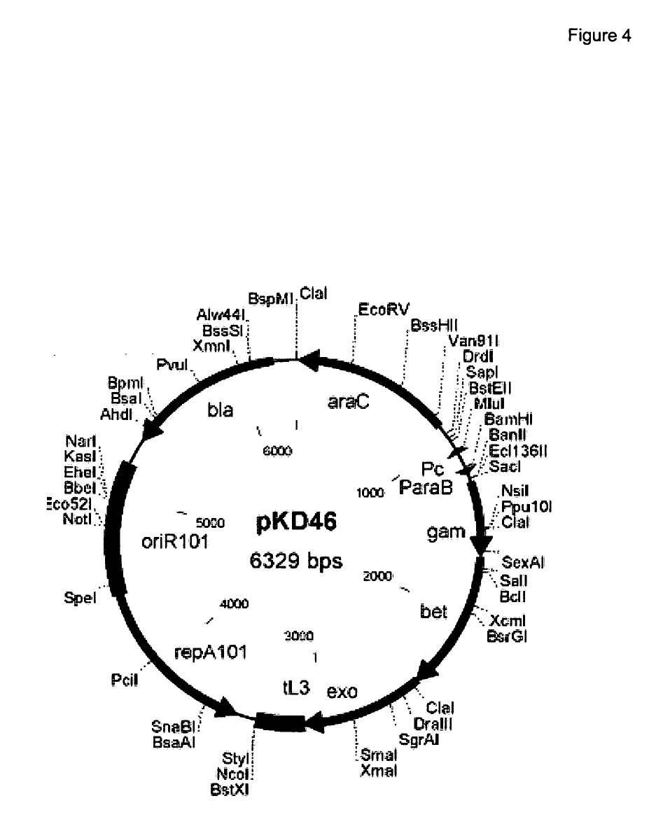

The λ-Red recombinase system used in the present invention is contained on a helper plasmid (pKD46) and is comprised of three essential genes, exo, bet, and gam (FIG. 4)(Datsenko and Wanner, supra). The exo gene encodes an λ-exonuclease, which processively degrades the 5′ end strand of double-stranded (ds) DNA and creates 3′ single-stranded overhangs. Bet encodes for a protein which complexes with the λ-exonuclease and binds to the single stranded DNA and promotes renaturation of complementary strands and is capable of mediating exchange reactions. Gam encodes for a protein that binds to the E. coli RecBCD complex, inhibiting endonuclease activity.

The λ-Red system is used in the present invention because homologous recombination in E. coli occurs at a very low frequency and usually requires extensive regions of homology. The λ-Red system facilitates the ability to use short regions of homology (10-50 bp) flanking linear dsDNA fragments for homologous recombination. Additionally, the RecBCD complex normally expressed in E. coli prevents the use of linear dsDNA for transformation as the complex's exonuclease activity efficiently degrades linear dsDNA. Inhibition of the RecBCD complex's endonuclease activity by gam is essential for efficient homologous recombination using linear dsDNA fragments.

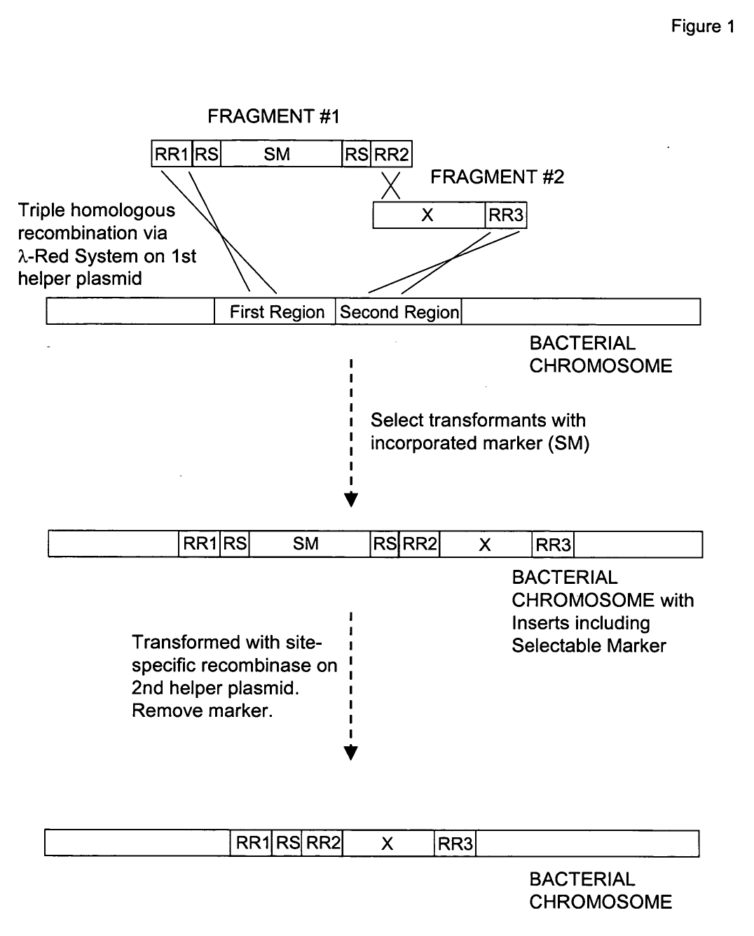

An important aspect of the present invention is the chromosomal promoter replacement in combination with a selection marker by using two PCR-generated, linear, double stranded DNA fragments. The invention involves the use of a triple homologous recombination event between two PCR-generated fragments and the E. coli chromosome (FIG. 1). The present method permits directed chromosomal engineering via triple homologous recombination without the need for a separate cloning step. The scope of the invention permits directed integration of functional DNA molecules (promoters, genes, regulatory elements, operons, etc.). The present method has no limitation in terms of chromosomal integration site and number. Therefore, the method is suitable for redesigning biosynthetic pathways, optimizing metabolic flux, and creating novel pathways.

Another aspect of the present method is the incorporation of selectable markers, such as antibiotic resistance, in one of the two linear double stranded DNA fragments. The selectable marker, such as one influencing antibiotic resistance, is used to select for those colonies that have undergone triple homologous recombination using the present invention. However, multiple chromosomal manipulations are needed when engineering bacterial biosynthetic pathways. In order to accomplish this, the method includes the use of site-specific recombination sequences flanking the selectable marker. Use of such site-specific recombinases to remove selectable markers is known in the art (Borges et al., WO 01/18222 A1; Bouhassira et al., WO 00/63410; and Martinez-Morales, et al., J. Bacteriol., 181:7143-7148 (1999)). Site-specific recombinases, such as the use of flippase (FLP) recombinase in the present invention, recognize specific recombination sequences (i.e. FRT sequences) and allow for the excision of the selectable marker. This aspect of the invention enables the repetitive use of the present method for multiple chromosomal modifications. The invention is not limited to the FLP-FRT recombinase system as several examples of site specific recombinases and their associated specific recognition sequences are know in the art. Examples of other suitable site-specific recombinases and their corresponding recognition sequences include, but are not limited to Cre/lox, Xer/dif, and Int/att.

Another aspect of the invention relates to its use in operon assembly on the bacterial chromosome by sequential integration of genes. Prokaryotic operons can be created using the present invention. An example of this aspect is illustrated in Example 13 where three carotenoid biosynthesis genes are integrated into the bacterial chromosome under the control of a single regulatory element (FIG. 13) via triple homologous recombination.

The present method allows for stable chromosomal integration of any promoter/regulatory region in front of any target gene in the E. coli chromosome so that the expression of such gene can be controlled. The method can also be used to engineer a variety of genetic elements, in addition to promoters, in the custom design of biosynthetic pathways. The approach is suitable for constructing industrially useful microbial strains, rather than just increased expression of a specific single gene. In terms of metabolic balance, productivity, control, stability, and optimal expression of the genes of a particular pathway, the approach has many advantages and benefits when compared to metabolic engineering based on a recombinant vector approach. The method is illustrated using E. coli as an example, but the method should prove to be useful in other bacterial strains as well (Poteete, A., supra, 2001). Examples of other industrially useful strains, which could be genetically engineered using the present invention, include Salmonella, Methylomonas, Pseudomonas, Bacillus and Acinetobacter.

Carotenoid Biosynthesis

Using several examples, the utility of the present method is illustrated by engineering increases in β-carotene (a carotenoid) production in an E. coli reporting strain. Carotenoids are pigments that are ubiquitous throughout nature and synthesized by all oxygen evolving photosynthetic organisms, and in some heterotrophic growing bacteria and fungi. Industrial uses of carotenoids include pharmaceuticals, food supplements, electro-optic applications, animal feed additives, and colorants in cosmetics, to mention a few.

Because animals are unable to synthesize carotenoids de novo, they must obtain them by dietary means. Thus, manipulation of carotenoid production and composition in plants or bacteria can provide new or improved source for carotenoids.

Carotenoids come in many different forms and chemical structures. Most naturally occurring carotenoids are hydrophobic tetraterpenoids containing a C₄₀ methyl-branched hydrocarbon backbone derived from successive condensation of eight C₅ isoprene units (IPP). In addition, novel carotenoids with longer or shorter backbones occur in some species of non-photosynthetic bacteria. The term “carotenoid” actually includes both carotenes and xanthophylls. A “carotene” refers to a hydrocarbon carotenoid. Carotene derivatives that contain one or more oxygen atoms, in the form of hydroxy-, methoxy-, oxo-, epoxy-, carboxy-, or aldehydic functional groups, or within glycosides, glycoside esters, or sulfates, are collectively known as “xanthophylls”. Carotenoids are furthermore described as being acyclic, monocyclic, or bicyclic depending on whether the ends of the hydrocarbon backbones have been cyclized to yield aliphatic or cyclic ring structures (G. Armstrong, (1999) In Comprehensive Natural Products Chemistry, Elsevier Press, volume 2, pp 321-352).

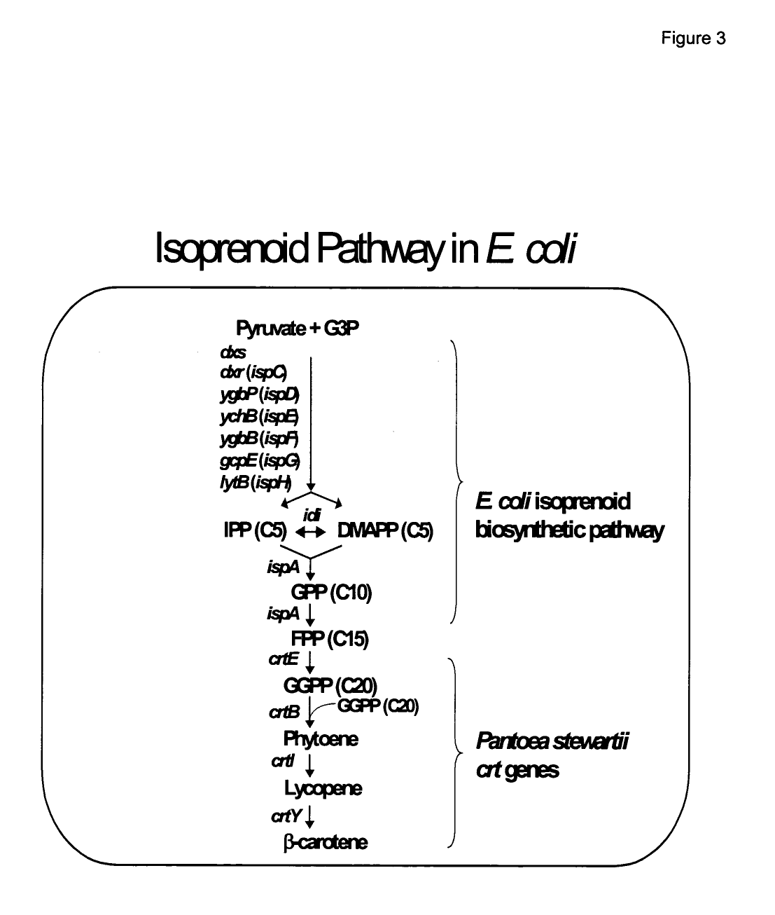

E. coli contains genes in the isoprenoid biosynthetic pathway (FIG. 3). Isoprenoid biosynthesis starts with the condensation of pyruvate with glyceraldehyde-3-phosphate (G3P) to form deoxy-D-xylulose via the enzyme encoded by the dxs gene. A host of additional enzymes are then used in subsequent sequential reactions, converting deoxy-D-xylulose to the final C5 isoprene product, isopentenyl pyrophosphate (IPP). IPP is converted to the isomer dimethylallyl pyrophosphate (DMAPP) via the enzyme encoded by the idi gene. IPP is condensed with DMAPP to form C10 geranyl pyrophosphate (GPP) which is then elongated to C15 farnesyl pyrophosphate (FPP).

FPP synthesis is common in both carotenogenic and non-carotenogenic bacteria. E. coli do not normally contain the genes necessary for conversion of FPP to β-carotene (FIG. 3). Because of this, the Applicant used an E. coli strain containing a reporter plasmid, pPCB15, which has the additional genes necessary for β-carotene production (FIG. 5). Enzymes in the subsequent carotenoid pathway used to generate carotenoid pigments from FPP precursor can be divided into two categories: carotene backbone synthesis enzymes and subsequent modification enzymes. The backbone synthesis enzymes include geranyl geranyl pyrophosphate synthase (CrtE), phytoene synthase (CrtB), phytoene dehydrogenase (CrtI) and lycopene cyclase (CrtY/L), etc. The modification enzymes include ketolases, hydroxylases, dehydratases, glycosylases, etc.

The genetics of carotenoid pigment biosynthesis are well known (Armstrong et al., J. Bact. 176: 4795-4802 (1994); Annu. Rev. Microbiol., 51:629-659 (1997)). This pathway is extremely well studied in the Gram-negative, pigmented bacteria of the genera Pantoea, formerly known as Erwinia. In both E. herbicola EHO-10 (ATCC 39368) and E. uredovora 20D3 (ATCC 19321), the crt genes are clustered in two operons, crt Z and crtEXYIB (U.S. Pat. No. 5,656,472; U.S. Pat. No. 5,5545,816; U.S. Pat. No. 5,530,189; U.S. Pat. No. 5,530,188; and U.S. Pat. No. 5,429,939). Despite the similarity in operon structure, the DNA sequences of E. uredovora and E. herbicola crt genes show no homology by DNA-DNA hybridization (U.S. Pat. No. 5,429,939,).

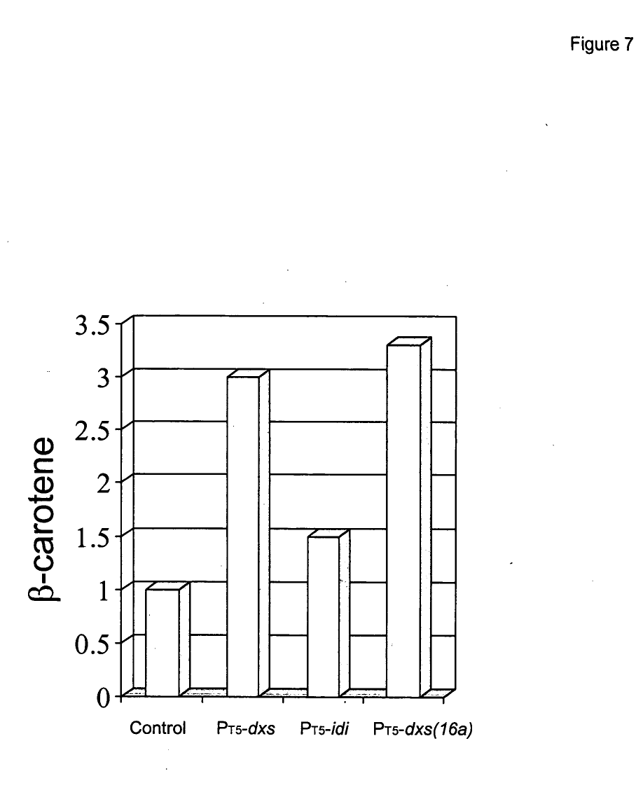

The E. coli dxs and idi genes encode for enzymes considered to be rate-controlling steps in the isoprenoid biosynthetic pathway (FIG. 3). The chromosomal copies of these genes have very low expression levels in the wild-type E. coli. The utility of the present invention is illustrated by integrating the phage T5 strong promoter (P_{T5}) in front of the dxs or idi genes resulting in increased the flux of the isoprenoid pathway. The result of the increased flux was detected as a measurable increase in β-carotene production (FIG. 7).

Recombination Elements

As used in the present invention, “recombination elements” are linear double-stranded DNA fragments or “cassettes” created using PCR. Typically the recombination elements will range from about 25 bases to about 4000 bases. The present method uses two recombination elements for in vivo bacterial chromosomal engineering. The linear elements contain functional DNA (regulatory elements, promoters, coding sequences, genes, etc.), optionally flanked by site-specific recombination sequences if the functional DNA is to be later removed as in the case of a selection marker. The 5′ and 3′ ends of the recombination elements are comprised of recombination regions (homology arms). The homology arms, generally about 10 to 50 base pairs in length, are chosen so have homology with either a specific sequence on the bacterial chromosome or a specific sequence on another recombination element.

The present invention illustrates the use of two recombination elements. As illustrated in FIG. 1, the 5′ end of the “first” recombination element contains a recombination region (“RR1”) having sequence homology to a targeted sequence on the bacterial chromosome. The 3′ end of the first element also contains a recombination region (“RR2”) having sequence homology to the 5′ end of the “second” recombination element. The 3′ end of the second recombination element (“RR3”) shares homology with a targeted sequence on the bacterial chromosome. During λ-Red mediated homologous recombination, the two elements are combined and inserted into a specific site on the bacterial chromosome based on the sequences of the homology arms. The actual sequence of the triple homologous recombination events may vary.

The preferred length of the homology arms is about 10 to about 50 base pairs in length. Given the relatively short lengths of the homology arms used in the present invention for homologous recombination, one would expect that the level of acceptable mismatched sequences should be kept to an absolute minimum for efficient recombination, preferably using sequences which are identical to those targeted for homologous recombination. From 20 to 40 base pairs of homology, the efficiency of homologous recombination increases by four orders of magnitude (Yu et al. PNAS. 97:5978-5983. (2000)). Therefore, multiple mismatching within homology arms may decrease the efficiency of homologous recombination. A large size (up to about 40 K base pairs) of the recombination elements can be PCR generated accurately by Taq DNA polymerase such as MasterAmp™ Extra-long PCR Enzyme (Epicentre, Inc. Madison, Wis.).

The present invention illustrates the use of a selectable marker (“SM” in FIG. 1) as the expressible DNA on one of the two recombination elements. The markers are flanked by site-specific recombinase recognition sequences (recognition target sequences, denoted as “RS” in FIG. 1). The recombination element containing the marker is usually not incorporated into the bacterial chromosome unless it is inserted simultaneously and in the correct orientation with the “second” recombination element. Expression of the selectable marker in the genetically modified bacteria is an indication that a triple homologous recombination event has probably occurred. After selection and construct verification, a site-specific recombinase is used to remove the marker. The steps of the present invention can then be repeated with additional in vivo chromosomal modifications. The use of selectable markers is know in the art and can include, but is not limited to, antibiotic resistance markers such as ampicillin, kanamycin, and tetracycline resistance. Selectable markers may also include amino acid biosynthesis enzymes (for selection of auxotrophs normally requiring the exogenously supplied amino acid of interest) and enzymes which catalyze visible changes in appearance such as β-galactosidase (catalyzes the conversion of Xgal into an easily discernable blue pigment) in lac⁻ bacteria.

Recombination Proficient Hosts

Recombination proficient hosts are hosts that contain a functional recombination system and are capable of efficiently being transformed by homologous recombination. In a preferred embodiment, the transformation via homologous recombination is targeted to the bacterial chromosome. In another preferred embodiment, the DNA used during the transformation process is PCR-generated, double-stranded DNA. E. coli, a host generally considered as one which does not undergo efficient transformation via homologous recombination naturally, is altered to make it a recombination proficient host. Transformation with a helper plasmid containing the λ-Red recombinase system increases the rate of homologous recombination several orders of magnitude. The λ-Red system can also be chromosomally integrated into the host. The λ-Red system contains three genes (exo, bet, and gam) which change the normally recombination deficient E. coli into a recombination proficient host.

Normally, E. coli efficiently degrades linear dsDNA via its RecBCD endonuclease, resulting in transformation efficiencies not useful for chromosomal engineering. Gam encodes for a protein that binds to the E. coli's RecBCD complex and blocks the complex's endonuclease activity. The exo gene encodes for a λ-exonuclease that processively degrades the 5′ end strand of double-stranded (ds) DNA and creates 3′ single stranded overhangs. The protein encoded by the bet gene complexes with the λ-exonuclease and binds to the single-stranded DNA overhangs and promotes renaturation of complementary strands and is capable of mediating exchange reactions. The λ-Red recombinase system allows one to use homologous recombination as a tool for in vivo chromosomal engineering in hosts, such as E. coli, normally considered difficult to transform by homologous recombination. The λ-Red system works in other bacteria as well (Poteete, A., supra, 2001). E. coli has been the primary focus of the λ-Red system because it is a commonly used industrial production host. There is no evidence that the λ-Red system should not be applicable to other hosts generally used for industrial production. These additional hosts include, but are not limited to Salmonella, Methylomonas, Pseudomonas, Bacillus, and Acinetobacter.

Transformation and Selection

The simple cloning bypass method described by the present invention allows one to conduct in vivo chromosomal engineering in a bacterial host. The method allows for repetitive targeted chromosomal modifications resulting in the ability to engineer biosynthetic pathways. The transformation process can be divided into several distinct steps.

First, recombination elements are PCR generated as previously described. A pair of dsDNA recombination elements is electroporated into a recombination proficient bacterial host. In the present invention, the host is E. coli MC1061 harboring a helper plasmid, pKD46, which contains the λ-Red recombinase system under the control of an arabinose inducible promoter. After electroporation, the host cells are cultured in the presence of arabinose. The λ-Red genes are expressed and triple homologous recombination occurs between the two recombination elements and the bacterial chromosome. One of the two chromosomally integrated recombination elements contains a selectable marker (kanamycin resistance; denoted as kan^{R} or kan).

After a period of time, the host cells are selected on a culture media containing a compound (i.e. kanamycin in the present invention) which selects for those cells that have undergone triple homologous recombination. In the present invention, a second resistance marker is use to confirm the presence of the pPCB15 reporter plasmid which contains a chloramphenicol resistance (Cam^{R}) gene. Colonies that grow in the presence of the selection medium are usually sequenced or analyzed by PCR fragment analysis to confirm the constructs.

After the confirmation of the constructs has occurred, a second helper plasmid is transfected into the host cell. The second “helper” plasmid contains a site-specific recombinase (Flp) which recognizes the site-specific recombinase target sequences (FRTs) flanking the chromosomally integrated selectable marker. The components expressed on both of the helper plasmids in the present invention could be chromosomally integrated into the host cell under the control of conditional promoters. The site-specific recombinase, Flp (flippase) in the present invention, is expressed and the selectable marker is excised. The colonies are cultured in the presence/absence of the selection compound to identify colonies that have lost the selection marker. The constructs of these colonies are confirmed by sequence analysis or by PCR fragment analysis or a combination of both. The second helper plasmid is cured from the host using a temperature-sensitive replication origin. The process described above can be repeated, allowing for targeted microbial pathway engineering and in the creation of industrially useful production hosts.

### DESCRIPTION OF THE PREFERRED EMBODIMENTS

An easy one-step method of bacterial in vivo chromosomal engineering has been developed using two linear double-stranded (ds), PCR-generated, DNA fragments. The fragments were designed to contain short flanking regions of homology between the fragments and the target site on the host (E. coli) chromosome.

The phage λ-Red recombinase system expressed on the helper plasmid pKD46 (GenBank® Accession number AY048746; SEQ ID NO:35; FIG. 4) and under control of an arabinose-inducible promoter was used, resulting in controllable and efficient in vivo triple homologous recombination. At least one of the two PCR-generated linear dsDNA fragments used during recombination was designed to contain a selective marker (kanamycin) flanked by site-specific recombinase sequences (FRT) (Example 1). The marker allowed for identification and selection of the cells that had undergone the desired recombination event. Once the constructs of the selected recombinants were verified by sequence analysis or PCR fragment analysis, the selective marker was excised by a second helper plasmid containing the site-specific recombinase gene under the control of the PR promoter of λ phage (Examples 3 and 4).

In one embodiment, the method was used for bacterial promoter engineering. A strong promoter (phage P_{T5}) was placed either upstream of the E. coli target gene dxs (Example 2) or idi (Example 6) via triple homologous recombination between two PCR-generated linear dsDNA fragments and the targeted chromosomal DNA (FIGS. 2 and 8). In each example, one of the two fragments contained a kanamycin resistance marker flanked by site-specific FRT recombinase sequences. Flanking the site-specific recombinase sequences were homology arms that contained short (approximately 10-50 bp) regions of homology. As illustrated in FIG. 2, a first recombination region (“h1”) was linked to the 5′-end of the first fragment. A second recombination region (“h2”) was linked to the 3′-end of the first fragment. The second PCR generated linear dsDNA fragment contained the P_{T5} strong promoter. The third recombination region (“h3”) was linked to the 3′-end of the second fragment. The first recombination region (“h1”) had homology to an upstream portion of the native bacterial chromosomal promoter targeted for replacement. The second recombination region (“h2”) located on the 3′-end of the first fragment had homology to the 5′-end portion of the second fragment. The third recombination region (“h3”) had homology to a downstream portion of the native bacterial chromosomal promoter targeted for replacement (FIG. 2).

The E. coli host, containing the λ-Red recombination system on the helper plasmid pKD46 (FIG. 4), was transformed with the two PCR-generated fragments resulting in the chromosomal replacement of the targeted native promoter with the construct containing the kanamycin selectable marker of first fragment and the P_{T5} strong promoter on the second fragment (Examples 4 and 7, FIGS. 2 and 8). The promoter replacement resulted in the formation of an augmented E. coli chromosomal gene (either dxs or idi gene), operably linked to the introduced non-native promoter. The bacterial host cells that had undergone the desired recombination event were selected according to the expression of the selectable marker and their ability to grow in selected media. The selected recombinants were then transformed with a second helper plasmid, pCP20 (ATCC PTA-4455; Datsenko and Wanner, supra), expressing the flippase (Flp) site-specific recombinase which excised the selectable marker (FIG. 2 and 8, Examples 3 and 6). The constructs were confirmed via PCR fragment analysis (FIGS. 6 and 9, Examples 4 and 7). The recombinant bacterial host cell containing the augmented gene (either dxs or idi) and the carotenoid reporter plasmid (pPCB15; SEQ ID NO:37) was then tested for increased production of β-carotene. Placement of either the E. coli dxs or idi genes, normally expressed at very low levels, under control of the P_{T5} strong promoter resulted in significant increases in β-carotene production (Example 5, FIG. 7).

In another embodiment, a reporter strain of E. coli was constructed for assaying β-carotene production. Briefly, the E. coli reporter strain was created by cloning the gene cluster crtEXYIB from Pantoea stewartii into a helper plasmid (pPCB15) which was subsequently used to transform the E. coli host strain (FIG. 5). Identification and selection for host cells containing the reporter plasmid was accomplished by culturing the cells in chloramphenicol (pPCB15 contains the chloramphenicol resistance gene, Cam^{R}). The cluster contains many of the genes required for the synthesis of carotenoids, with β-carotene normally produced in the transformed E. coli (FIG. 7). It should be noted that the crtZ gene (β-carotene hydroxylase) was included in the gene cluster. However, since no promoter was present to express the crtZ gene (organized in opposite direction and adjacent to crtB gene) no zeaxanthin was produced. Thus, the zeaxanthin glucosyl transferase enzyme (encoded by the crtX gene located within the gene cluster) had no substrate for its reaction. Increases in β-carotene production were reported as increases relative to the control strain production (FIG. 7).

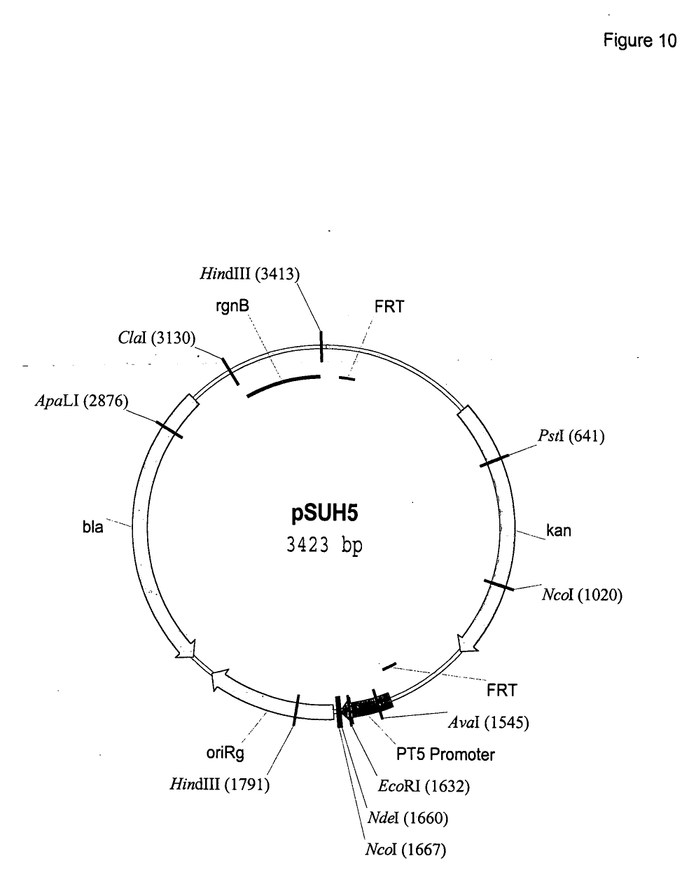

The general method can be used for a variety of chromosomal modifications based on the genetic information contained on the two PCR generated fragments. In another embodiment, the method was used to simultaneously add a foreign gene and promoter. The first of the two PCR-generated fragments was designed so that is contains the fusion product of a selectable marker (kanamycin) and promoter (P_{T5}) (Example 8, FIGS. 10 and 11)). The second PCR-generated fragment contained a foreign gene, the Methylomonas 16a strain dxs gene denoted as “dxs(16a)”. Once again, homology arms were designed to allow for precise incorporation into the host bacterial chromosome. The gene of interested can be simply added or used to replace a targeted native gene depending upon the selected homology arms used. The desired recombinants were selected by methods previously described. The selectable marker was then removed by a site-specific recombinase as previously described (FIG. 11, Example 9). The recombinant constructs were confirmed by PCR fragment analysis (FIG. 12, Example 10). The effect on β-carotene production in the transformed E. coli reporter strain was measured as previously described. Cells containing the Methylomonas 16a strain dxs gene (homologous to the E. coli dxs gene) under the control of the P_{T5} strong promoter exhibited significant increases in β-carotene production (FIG. 7). The present one-step method using homologous recombination was useful in the simultaneous addition of a foreign promoter and gene. Subsequent removal of the selectable marker is required so that the process can be repeated, if desired, to engineer bacterial biosynthetic pathways so that the final result is increased production of the desired product.

In another embodiment, the applicability of the present method was illustrated by the creation of an E. coli operon. The prokaryotic operon created was comprised of three Pantoea stewardii carotenoid biosynthesis genes (crtE, crtI, and crtB genes) operably linked to a single strong promoter (P_{T5}) The first PCR-generated linear dsDNA fragment was comprised of a fusion product between a kanamycin selectable marker, a P_{T5} strong promoter, and a gene of interest (Pantoea stewartii crtE) operably linked to the P_{T5} promoter (kan-P_{T5}-crtE, FIG. 13). The kanamycin selectable marker was flanked by site-specific recombinase sites for later removal as previously described. The first fragment was flanked by appropriate homology arms on the 5′ and 3′-ends. A second PCR-generated linear dsDNA fragment was constructed to contain the foreign P. stewartii crtIB genes and contained an appropriate 3′-end homology arm (FIG. 13, Example 12). Triple homologous recombination occurred as previously described resulting in the chromosomal integration and formation of an operon containing the crtE gene of first fragment closely associated with the crtIB genes of the second fragment, operably linking all of the genes to the P_{T5} promoter on the first fragment. Once again, the marker was removed after selection in an appropriate media as previously described. The constructs were confirmed by PCR fragment analysis (FIG. 14, Example 13). The transformed E. coli strain containing the functionally expressed crtEIB operon did not contain the pPCB15 reporter plasmid as in previous examples. It should be noted that the pPCB15 reporter plasmid also contained a functionally expressed crtY gene (lycopene cyclase) which was responsible for converting lycopene (pink color) into β-carotene (orange color). Expression of the crtEIB operon in the transformed E. coli strain lacking the pPCB15 helper plasmid was confirmed by the accumulation of pink colonies (indicative of lycopene accumulation) (Example 13).

In another embodiment, a bacterial host strain can be engineered to contain multiple chromosomal modifications, including multiple promoter and gene additions or replacements so that the production efficiency of the desired final product is increased. In a preferred embodiment, the incorporated or augmented chromosomal genes encode for enzymes useful for the production of carotenoids. In a further preferred embodiment, multiple copies of genes may be chromosomally engineered into the host genome.

The present one-step method can be used for a variety of targeted in vivo bacterial chromosomal modifications. The genetic information contained within the two PCR-generated dsDNA fragments (and their associated homology arms) determines the type and location of the desired chromosomal modification. The elimination of a cloning step used in previous methods of transformation eliminates a lot of laboratory time and its associated expenses. The removal of the selectable marker using a site-specific recombinase allows for one to conduct multiple chromosomal modifications, necessary for engineering biosynthetic pathways and for optimizing production of industrially useful materials.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

General Methods

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Manipulations of genetic sequences were accomplished using the suite of programs available from the Genetics Computer Group Inc. (Wisconsin Package Version 9.0, Genetics Computer Group (GCG), Madison, Wis.). Where the GCG program “Pileup” was used the gap creation default value of 12, and the gap extension default value of 4 were used. Where the CGC “Gap” or “Bestfit” programs were used the default gap creation penalty of 50 and the default gap extension penalty of 3 were used. Multiple alignments were created using the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.). In any case where program parameters were not prompted for, in these or any other programs, default values were used.

The meaning of abbreviations is as follows: “h” means hour(s), “min” means minute(s), “sec” means second(s), “d” means day(s), “mL” means milliliter(s), “μL” means microliter(s), “L” means liter(s), and “rpm” means revolutions per minute.

λ-Red Recombinase Plasmid System

The plasmids (pKD4, pKD46 and pCP20) used in the present invention have been previously described in the literature (Datsenko and Wanner, supra). The sequences for the λ-Red recombinase system components are contained on helper plasmid pDK46 (SEQ ID NO:35; GenBank® Accession No. AY048746). The Flp/FRT site-specific recombinase helper plasmid used in the present invention was pCP20 (ATCC Number PTA-4455) Plasmid pKD4 (SEQ ID NO:34; GenBank® Accession No. AY048743) was used as a template molecule for PCR amplification of the FRT flanked kanamycin resistance marker).

### Example 1

Synthesis of Two Linear PCR-Generated DNA Fragments used for Engineering the Promoter of dxs Gene on the E. coli Chromosome

The linear DNA fragment (1489 bp) containing a kanamycin selectable marker flanked by site-specific recombinase target sequences (FRT) was synthesized by PCR from plasmid pKD4 (Datsenko and Wanner, supra) with primer pairs, T1(dxs) (5′-TGGAAGCGCTAGCGGACTACATCATCCAGCGTAATAATAACGTCTT GAGCGATTGTGTAG-3′) (SEQ ID NO:1) which contains a h1 homology arm (underlined, 41 bp) chosen to match sequences in the upstream region of the ispA stop codon and a priming sequence (20 bp) and B1(T5) (5′-CTCGAGGTGAAGACGAAAGGGCCTCGTGATACGCCTATTTTTATA GGTTACATATGAATATCCT CCTTAG -3′) (SEQ ID NO:2) that contains a h2 homology arm (underlined, 50 bp) chosen to match sequences in the 5′-end region of the promoter DNA fragment and a priming sequence (20 bp) (FIG. 2). A second linear DNA fragment (154 bp) containing a phage promoter (P_{T5}) comprised of the −10 and −35 consensus sequences, lac operator (lacO), and ribosomal binding site (rbs) was synthesized by PCR from plasmid pQE30 (Qiagen, Valencia, Calif.) with primer pairs, T2(T5) (5′-TAACCTATAAAAA TAGGCGTATCACGAGG CCC-3′) (SEQ ID NO:3) that contains a priming sequence (32 bp) and B2(dxs) (5′-GGAGTCGACCAG TGCCAGGGTCGGGTATTTGG CAATATCAAAAC TCATAGTTAATTTCTCCTCTTTAATG-3′) (SEQ ID NO:4) that contains a h3 homology arm (underlined, 48 bp) chosen to match sequences in the downstream region of the dxs start codon and a priming sequence (22 bp) (FIG. 2). The underlined sequences illustrate each respective homology arm, while the remainder is the priming sequences for hybridization to complementary nucleotide sequences on the template DNA for the PCR reaction. The two resultant PCR fragments were the kanamycin selectable marker containing the homology arms (h1 and h2) and the P_{T5} promoter containing the homology arm (h3) as illustrated in FIG. 2. Standard PCR conditions were used to amplify the linear DNA fragments with Ampli Taq Gold® polymerase (Applied Biosystems, Foster City, Calif.) as follows;

| PCR reaction: | PCR reaction mixture: |
|---|---|
| Step1 94 ° C. 3 min | 0.5 μL plasmid DNA |
| Step2 93 ° C. 30 sec | 5 μL 10X PCR buffer |
| Step3 55 ° C. 1 min | 1 μL dNTP mixture (10 mM) |
| Step4 72 ° C. 3 min | 1 μL 5′-primer (20 μM) |
| Step5 Go To Step2, 30 cycles | 1 μL 3′-primer (20 μM) |
| Step6 72 ° C. 5 min | 0.5 μL Taq DNA polymerase |
| | 41 μL sterilized dH₂O |

After completing the PCR reactions, PCR products were purified using the QlAquick Gel Extraction Kit™ (Cat. # 28704, QIAGEN Inc. Valencia, Calif.). Briefly, 50 μL of each PCR reaction mixture was run on a 1% agarose gel. The band containing the amplified DNA fragment was excised from the agarose gel with a clean, sharp scalpel. The gel slice was weighted in a colorless plastic tube. After adding 3 volumes of buffer QG to 1 volume of gel, the tube was incubated at 50° C. for 10 min (or until the gel slice has completely dissolved). After the gel slice has dissolved completely, one gel volume of isopropanol was added to the sample and mixed by inverting the tube several times. In order to bind DNA, the sample was applied to the QlAquick gel extraction column and centrifuged for 1 min. 750 μL of buffer PE was applied to the column for washing. After centrifuging for 1 min to remove the wash buffer, PCR DNA products were eluted with 10 μL of distilled water (dH₂O) by standing sample for 1 min, and then by centrifuging for 1 min. DNA Clean & Concentrator™ (Zymo Research, Orange, Calif.) was used to further purify the PCR DNA samples. After adding 2 volumes of DNA Binding Buffer to each volume of DNA sample, the samples were loaded into a Zymo-Spin Column (Zymo Research) and centrifuged at full speed (>10,000 g) for 5-10 sec. The PCR DNA sample retained in the column was washed twice with 200 μL of Wash Buffer by centrifuging at top speed for 5-10 seconds. The DNA was eluted with 6-8 μL of distilled water by spinning at top speed two times. The concentration of PCR DNA sample was about 0.5-1.0 μg/μL.

### Example 2

Electro-Transformation of Two Linear PCR-Generated DNA Fragments into an E. coli Strain Expressing λ-Red Recombinase

The E. coli MC1061 strain, carrying both a λ-Red recombinase expression plasmid (FIG. 4) and a reporter plasmid (FIG. 5), was used as a host strain for the recombination of PCR fragments. The strain was constructed by the co-transformation with a λ-Red recombinase expression plasmid, pKD46 (amp^{R}) (Datsenko and Wanner, supra) (FIG. 4) and a β-carotene biosynthesis expression plasmid, pPCB15 (cam^{R}) (FIG. 5) into the E. coli strain MC1061. The plasmid pPCB15 contains the carotenoid gene cluster (crtEXYIB) from Pantoea Stewartii (ATCC 8199). The λ-Red recombinase in pKD46 is comprised of three genes exo, bet, and gam expressed under the control of an arabinose-inducible promoter. Transformants were selected on 100 μg/mL ampicillin and 25 μg/mL chloramphenicol LB plates at 30° C.

The electro-competent cells of E. coli MC1061 strain carrying pKD46 and pPCB15 were prepared as follows. E. coli MC1061 cells carrying pKD46 and pPCB15 were grown in SOB medium with 100 μg/mL ampicillin, 25 μg/ml chloramphenicol, and 1 mM L-arabinose at 30° C. to an OD₆₀₀ of 0.5, following by chilling on ice for 20 min. Bacterial cells were centrifuged at 4,500 rpm using a Sorvall® RT7 PLUS (Kendro Laboratory Products, Newton, Conn.) for 10 min at 4° C. After decanting the supernatant, the pellet was resuspended in ice-cold water and centrifuged again. This was repeated twice and the cell pellet was resuspended in 1/100 volume of ice-cold 10% glycerol.

Both the kanamycin marker PCR products (1-5 μg) and phage P_{T5}promoter PCR products (1-5 μg) were mixed with 50 μL of the competent cells and pipetted into a pre-cooled electroporation cuvette (0.1 cm) on ice. Electroporation was performed by using a Bio-Rad Gene Pulser set at 1.8 kV, 25 μF with the pulse controller set at 200 ohms. SOC medium (1 mL) was added after electroporation. The cells were incubated at 37° C. for 1 hour. Approximately one-half of cells were spread on LB plates containing both 25 μg/mL kanamycin and 25 μg/mL chloramphenicol in order to select double antibiotic-resistant transformants. After incubating the plate at 37° C. overnight, one double antibiotic-resistant transformant was selected.

### Example 3

Elimination of the Antibiotic Selectable Marker Using a FLP Recombinase Expression Plasmid

The Flp recombinase expression plasmid pCP20 (amp^{R}), which has a temperature-sensitive replication origin, was transiently transformed into the kanamycin-resistant transformant by electroporation. Bio-Rad Gene Pulser (Bio-Rad Laboratories, Hercules, Calif.) set at 1.8 kV, 25 μF with the pulse controller set at 200 ohms was used for electro-transformation. Cells were spread on 100 μg/mL ampicillin and 25 μg/mL chloramphenicol LB plates, and grown at 30° C. for 1 day. Colonies were picked and streaked on 25 μg/mL chloramphenicol LB plates without ampicillin antibiotics and incubated at 43° C. overnight (Plasmid pCP20 has a temperature sensitive origin of replication and was cured from the host cells by culturing cells at 43° C.). The colonies were tested for ampicillin and kanamycin resistance to test loss of pCP20 and kanamycin selectable marker by streaking colonies on 100 μg/mL ampicillin LB plate or 25 μg/mL kanamycin LB plate. The kanamycin selectable marker was removed by Flp recombination between the FRT sites by transient transformation of a Flp expression plasmid based on the temperature-sensitive origin.

### Example 4

Confirmation of Chromosomal Integration of the Phage P_{T5} Promoter in Front of the dxs Gene by PCR Analysis

The chromosomal integration of both the kanamycin selectable marker and phage P_{T5} promoter in the front of dxs gene on E. coli chromosome was conformed by PCR analysis. A colony of transformants was resuspended in 50 μL of PCR reaction mixture containing 200 μM dNTPs, 2.5 U AmpliTaq Gold™ (Applied Biosytems), 0.4 μM of different primer pairs, T1 (5′-CGCCAGTCGCTGAAACAACTGGCTGA-3′)(SEQ ID NO:5) and T2 (5′-CAGTCATAGC CGAATAGCCT-3′)(SEQ ID NO:6), T3 (5′-CGGTGCCCTGAATGAACTGC-3′)(SEQ ID NO:7) and T4 (5′-TGGCAACA GTCGTAGCTCCTGGGTGG-3′) (SEQ ID NO:8), T2(T5) and T4, and T1 and T4. Test primers were chosen to amplify regions located either in the vicinity of the integration region or in the kanamycin and phage P_{T5} promoter region (FIG. 6). PCR test with T1 and T2, T3 and T4, T2(T5) and T4, and T1 and T4 primer pairs revealed the expected sizes, 682 bp, 1116 bp, 229 bp and 1887 bp on 1% agarose gel, respectively (FIG. 6). PCR analysis was repeated after elimination of the kanamycin selectable marker, and showed no PCR products for T1 and T2, and T3 and T4, and the expected sizes 229 bp and 494 bp for T2(T5)/T4 and T1/T4, respectively (FIG. 6). The PCR results indicated the correct integration of the P_{T5} promoter fragment in the front of chromosomal dxs gene, yielding E. coli P_{T5}-dxs.

### Example 5

Measurement of β-Carotene Production in E. coli P_{T5}-dxs

The chromosomal integration of the P_{T5} promoter in the front of dxs gene was further characterized by measuring the β-carotene production of E. coli P_{T5}-dxs containing the reporter plasmid, pPCB15, along with E. coli control strain also containing pPCB15. E. coli P_{T5}-dxs and E. coli control strains were grown in 5 mL LB containing 25 μg/mL chloramphenicol at 37° C. for 24 h, and then harvested by centrifugation at 4000 rpm for 10 min. The β-carotene pigment was extracted by resuspending the cell pellet in 1 mL of acetone with vortexing for 1 min and then shaking the sample for 1 h at room temperature by using Rocking Platform 200 (VWR International, West Chester, Pa.). After centrifuging the sample at 4000 rpm for 10 min, the absorption spectrum of the acetone layer containing β-carotene was measured at λ 450 nm by using Ultrospec 3000 spectrophotometer (Amersham Biosciences, Piscataway, N.J.). The production of β-carotene in E. coli P_{T5}-dxs was approximately 3-fold higher than the control strain (FIG. 7). The results indicate that the increased production of β-carotene in E. coli P_{T5}-dxs was due to the increased expression of the dxs gene under control of the strong P_{T5} promoter. The E. coli P_{T5}-dxs construct was shown to be correct by testing its function from the measurement of β-carotene in E. coli P_{T5}-dxs versus control strain as well as by PCR analysis.

### Example 6

Engineering the Promoter of the idi Gene on the E. coli Chromosome—Preparation of Two Linear (PCR-Generated) DNA Fragments, Electro-Transformation, and Elimination of the Antibiotic Selectable Marker

The general applicability of this invention to the chromosomal promoter replacement was further examined by engineering the promoter of idi gene on E. coli chromosome. The endogenous promoter, operably linked to idi gene located at 65.3 min of the E. coli chromosome, was replaced with a P_{T5} strong promoter by using two PCR-generated DNA fragments. The DNA fragments were synthesized by PCR as described in Example 1, except using different primers. The linear DNA fragment (1489 bp) containing a kanamycin selectable marker flanked by FRT sequences was synthesized by PCR from plasmid pKD4 (Datsenko and Wanner, supra) with primer pairs, T(idi) (5′-TCTGATGCGCAAGCCTGAAGAAAAATGAGCATGGAGAATAATATGACG TCTTGAGCGATTGTGTAG-3′)(SEQ ID NO:9) which contains a h4 homology arm (underlined, 45 bp) chosen to match a sequence in the upstream region of the ygfU stop codon and a priming sequence (20 bp) and B1(T5) that contains a h2 homology arm (underlined, 50 bp) chosen to match sequences in the 5′-end region of the promoter DNA fragment and a priming sequence (20 bp) (FIG. 8). A second linear DNA fragment (154 bp) containing a P_{T5} promoter was synthesized by PCR from pQE30 (QIAGEN, Inc.) with primer pairs, T2(T5) (5′-TAACCTATAAAAA TAGGCGTATCACGAGG CCC-3′) (SEQ ID NO:3) that contains a priming sequence (32 bp) and B(idi) (5′-TGGGAACTCCCTGTGCATTCAATAAAATGACGTGTTCCGTTTG CATAGTTAATTTCTCCTCTTTAATG-3′)(SEQ ID NO:10) which contains a h5 homology arm (underlined, 46 bp) chosen to match sequences in the downstream region of the idi start codon and a priming sequence (22 bp) (FIG. 8). The underlined sequences illustrate each respective homology arm, while the remainder is the priming sequences for hybridization to complementary nucleotide sequences on the template DNA for the PCR reaction. The two resultant PCR fragments were the kanamycin selectable marker containing the homology arms (h2 and h4) and the P_{T5}promoter containing the homology arm (h5) as illustrated in FIG. 8.

The PCR reaction, purification, and electro-transformation were performed as described in Example 1 and 2. The purified kanamycin cassette PCR products (1-5 μg) and P_{T5} promoter PCR products (1-5 μg) were co-transformed into E. coli expressing λ-Red recombinase. Cells were plated and screened for antibiotic-resistant transformants as described in Example 1. Fourteen kan^{R} and cam^{R} double-resistant transformants were selected. The kanamycin selectable marker was eliminated according to the method as described in Example 3.

### Example 7

Confirmation of Chromosomal Integration of the P_{T5} Promoter in Front of the idi Gene

Among fourteen double antibiotic-resistant transformants, five transformants were arbitrarily selected and were analyzed by PCR analysis with different combination of specific primer pairs, T5 (5′-TCATGCTGACCTGGTGAAGGAATCC-3′)(SEQ ID NO:11) and T2, T2(T5) and T6 (5′-TCATGCTGACCTGGTGAAGGAATCC-3′)(SEQ ID NO:12), and T5 and T6 (FIG. 9). The PCR reaction was performed as described in Example 4. Test primers were chosen to amplify sequences located either in the vicinity of the integration region or in the kanamycin and the P_{T5} promoter region. PCR test with T5 and T2, T2(T5) and T6, and T5 and T6 primer pairs for all five transformants gave the expected sizes, 627 bp, 274 bp and 1877 bp on 1% agarose gel, respectively. After elimination of the kanamycin selectable marker as described in Example 3, PCR analysis with T5 and T2 showed no PCR products. PCR analysis with T2(T5) and T6 and T5 and T6 primer pairs was repeated and showed PCR products with the expected sizes or 274 bp and 484 bp, respectively. The results indicated the correct integration of the kanamycin selectable marker and the P_{T5} promoter fragment in the front of chromosomal idi gene, yielding E. coli P_{T5}-idi.

The chromosomal integration of a P_{T5} promoter in the front of idi gene was further confirmed by quantification of the β-carotene production of E. coli P_{T5}-idi versus control strain as described in Example 5. The E. coli P_{T5}-idi exhibited a 1.5-fold increase in the production of β-carotene when compared to the control strain containing the idi gene linked to its native promoter (FIG. 7).

### Example 8

Construction of a Template Plasmid Carrying a Fused Antibiotic Selectable Marker and Promoter

The plasmid carrying a fused kanamycin selectable marker and P_{T5} promoter was constructed to facilitate the chromosome engineering. The plasmid containing a fused kanamycin selectable marker and the P_{T5} promoter was constructed by cloning a P_{T5} strong promoter region into the Ndel restriction endonuclease site of pKD4 (Datsenko and Wanner, supra) (FIG. 10). First, the linear DNA fragment containing the P_{T5} promoter element (154 bp) comprised of the −10 and −35 consensus sequences, lacO, and rbs was synthesized by PCR from pQE30 (QIAGEN, Inc.) with primer pairs, TT5 (5′-ATCATGACATTAACATATGAAAATAGGCGTATCACGAGGCCC-3′) (SEQ ID NO:13) and BT5 (5′-GATGCGATCCCATATGAGTTAATTTCTCCTCTTTAATG-3′) (SEQ ID NO:14). The underlined sequences in the primers represent the sequence of the Ndel site. The PCR reaction condition was same as described in Example 1. The resulting PCR product was digested with Ndel and cloned into Ndel-digested pKD4, yielding pSUH5 (SEQ ID NO:36; FIG. 10). The plasmid pSUH5 was used as a template to generate the linear PCR DNA fragment containing the fused kanamycin selectable marker-phage P_{T5} promoter. The pSUH5 construction was confirmed by DNA sequencing (DuPont CR&D DNA sequence facility, Wilmington, Del.).

### Example 9

Chromosomal Integration of the Methylomonas 16A dxs Gene in E. coli—Preparation of Two Linear DNA Fragments: (1) Fused Antibiotic Selectable Marker-Promoter and (2) Foreign Gene, Electro-Transformation and Elimination of the Antibiotic Selectable Marker

The applicability of this invention was further extended to chromosomal integration of a foreign gene into E. coli. This example describes the chromosomal integration of the Methylomonas 16a dxs gene (denoted as “dxs(16a)” in the present invention; SEQ ID NO:15) into the region between genes located at 30.9 min of E. coli chromosome by homologous recombination between the E. coli chromosome and two linear DNA fragments, a fused kanamycin selectable marker-phage P_{T5} promoter and the Methylomonas 16a dxs gene. The dxs gene is the Methylomonas 16a (ATCC PTA-2402) homologue of the E. coli dxs gene. The scheme for the chromosomal integration of the Methylomonas 16a dxs gene by homologous recombination is shown in FIG. 11.

The linear DNA fragment containing fused kanamycin selectable marker-phage P_{T5} promoter was synthesized by PCR from pSUH5 with primer pairs, T1(dxs16a) (5′-CACTAACGCCCGCACATTGCTGCGGGCTTTTTGATTCATTTCGCACG TCTTGAGCGATTGTGTAG-3′)(SEQ ID NO:16) which contains a h6 homology arm (underlined, 45 bp) chosen to match a sequence in the region between genes located at 30.9 min of E. coli chromosome and a priming sequence (20 bp) and B1(dxs16a) (5′-AGTAGAGGGAAGTCTTTGGAAAGAGCCATAGTTAATTTCTCCTCTTTA ATG-3′)(SEQ ID NO:17) which contains a h7 homology arm (underlined, 29 bp) chosen to match a sequence in the downstream region of the dxs(16a) start codon and a priming sequence (22 bp) (FIG. 11). The linear DNA fragment containing the Methylomonas 16a dxs gene was synthesized by PCR from Methylomonas 16a (ATCC PTA-2402) genomic DNA with primer pairs, T2(dxs16a) (5′-ACAGAATTCATTAAAGAGGAGAAATTAACTATGGCTCTTTCCAAAGAC TTCCCTC-3′)(SEQ ID NO:18) which contains a h8 homology arm (underlined, 30 bp) chosen to match a sequence in the 3′-end region of the fused kanamycin selectable marker-P_{T5} promoter and a priming sequence (25 bp) and B2(dxs16a) (5′-AGGAGCGAAGTGATTATCAGTATGCTGTTCATATAGCCTCGAATTATC AAGCGCAAAACTGTTCGATG-3′)(SEQ ID NO:19) which contains a h9 homology arm (underlined, 46 bp) chosen to match a sequence in the region between genes located at 30.9 min of the E. coli chromosome and a priming sequence (22 bp) (FIG. 11). The underlined sequences illustrate each respective homology arm, while the remainder is the priming sequences for hybridization to complementary nucleotide sequences on the template DNA for the PCR reaction. The two resultant PCR fragments were the kanamycin selectable marker containing the homology arms (h6 and h7) and the dxs(16a) gene containing the homology arms (h8 and h9) as illustrated in FIG. 11.

The PCR reaction, purification and electro-transformation were performed as described in Example 1 and 2. Both the fused kanamycin-phage P_{T5} promoter PCR products (1-5 μg) and the Methylomonas 16a dxs PCR products (1-5 μg) were co-transformed into an E. coli host cell expressing the λ-Red recombinase system. Transformants were selected as previously described and seven kan^{R} and cam^{R} double-resistant transformants were selected. The kanamycin selectable marker was eliminated according to the method as described in Example 3.

### Example 10

Confirmation of Chromosomal Integration of the Fused Antibiotic Selectable Marker-Promoter and the Foreign dxs Gene from Methylomonas 16A.

All seven double antibiotic-resistant transformants were PCR-analyzed with different combination of specific primer pairs, T7 (5′-ACCGGATATCACCACTTAT CTGCTC-3′)(SEQ ID NO:20) and T2, T7 and T8 (5′-ATGCTGACCGTGTGGGTCAGATAGC-3′)(SEQ ID NO:21), T2(T5) and T9 (5′-GCGATATTGTATGTCTGATTCAGGA-3′)(SEQ ID NO:22), and T7 and T9 (FIG. 12). Test primers were chosen to amplify sequences located either in the vicinity of the integration region of the kanamycin selectable marker-P_{T5} promoter region or the Methylomonas 16a dxs gene as shown in FIG. 12. The PCR reaction was performed as described in Example 4. From the PCR analysis with pairs T7 and T2, T7 and T8, T2(T5) and T9, and T7 and T9, two out of seven transformants gave the expected sizes, 643 bp, 1908 bp, 2184 bp and 3803 bp on 1% agarose gel, respectively. The results indicated that the two transformants had the correct integration of the fused kanamycin selectable marker-P_{T5} promoter fragment and the Methylomonas 16a dxs gene into the region between genes located at 30.9 min of E. coli chromosome. After elimination of the kanamycin selectable marker, PCR analysis with primer pair T7 and T2 resulted in no PCR product. PCR analysis with primer pairs T7 and T8, T2(T5) and T9, and T7 and T9 showed the expected size fragments of 515 bp, 2184 bp and 2410 bp on 1% agarose gel, respectively. The resultant E. coli P_{T5}-dxs(16a) transformation was tested for its β-carotene production as described in Example 5 and exhibited 3.3-fold higher in the production of β-carotene than control strain (FIG. 7).

### Example 11

Chromosomal Integration of the P. stewartii crtE Gene in E. coli—Preparation of Two Linear DNA Fragments: (1) Fused Kanamycin Selectable Marker-P_{T5} Promoter and (2) the P. stewartii crtE Gene.

This example describes the chromosomal integration of P. stewartii crtE and crtIB genes into the inter-operon region located at 81.2 min of E. coli chromosome by integration of P. stewartii crtE and P. stewartii crtIB (FIG. 13). The crtE, crtI, and crtB genes encode geranylgeranyl pyrophosphate synthase, phytoene dehydrogenase, and phytoene synthase, respectively. These genes are part of the carotenoid biosynthetic pathway (FIG. 3).

The linear DNA fragment containing fused kanamycin selectable marker-P_{T5} promoter is synthesized by PCR from pSUH5 with primer pairs, T1(crtE) (5′-AGCCGTCGCAGGAGGAACAACTCATATCATCATTGCGATCTCGACCG TCTTGAGCGATTGTGTAG-3′)(SEQ ID NO:23) which contains a h10 homology arm (underlined, 45 bp) chosen to match a sequence in the inter-operon region located at 81.2 min of E. coli chromosome and a priming sequence (20 bp) and B1(crtE) (5′-TGAACGTGTTTTTTTGCGCAGACCGTCATAGTTAATTTCTCCTCTTTA ATG-3′)(SEQ ID NO:24) which contains an h1 homology arm (underlined, 29 bp) chosen to match a sequence in the downstream region of the crtE start codon and a priming sequence (22 bp) (FIG. 13). The linear DNA fragment containing P. stewartii crtE gene was synthesized by PCR from pPCB15 (FIG. 5) with primer pairs, T2(crtE) (5′-ACAGAATTCATTAAAGAGGAGAAATTAACTATGACGGTCTGCGCAAA AAAACACG-3′)(SEQ ID NO:25) which contains an h8 homology arm (underlined, 30 bp) chosen to match a sequence in the 3′-end region of the fused kanamycin selectable marker-P_{T5} promoter and a priming sequence (25 bp) and B2(crtE) (5′-AGAATGACCAGCTGGATGCATTATCTTTATTTGGATCATTGAGGGTTA ACTGACGGCAGCGAGTT-3′)(SEQ ID NO:26) which contains an h12 homology arm (underlined, 45 bp) chosen to match a sequence in the inter-operon region located at 81.2 min of the E. coli chromosome and a priming sequence (20 bp) (FIG. 13). The underlined sequences illustrate each respective homology arm, while the remainder is the priming sequences for hybridization to complementary nucleotide sequences on the template DNA for the PCR reaction. The two resultant PCR fragments were the fused kanamycin selectable marker-P_{T5} promoter containing the homology arms (h10 and h11) and the P. stewartii crtE gene containing the homology arms (h8 and h12) as illustrated in FIG. 13.

The PCR reaction, purification, and electro-transformation were performed as described in Example 2 except that the transformation of the reporter plasmid pPCB15 into E. coli strain was omitted. As noted previously, plasmid pPCB15 contains the Pantoea stewartii (ATCC 8199) crtEXYIB gene cluster. In order to measure for functional expression of any of the genes chromosomally engineered into E. coli as described in Examples 12 and 13, it was necessary to omit the reporter plasmid. Both fused kanamycin marker-P_{T5} promoter PCR products (1-5 μg) and the P. stewartii crtE PCR products (1-5 μg) were co-transformed into an E. coli host strain (MC1061) expressing the λ-Red recombinase system by electroporation as previously described in Example 2 except for the omission of the reporter plasmid as described above. Transformants were selected on 25 μg/mL kanamycin LB plates at 37° C. After incubating the plate at 37° C. overnight, two kan^{R} resistant transformants were selected.

Two kan^{R} resistant transformants were PCR analyzed with T10 (5′-CCATGACCCTACATTGTGATCTATAG-3′)(SEQ ID NO:27 and T13 (5′-GGAACCATTGAACTGGACCCTAACG-3′)(SEQ ID NO:33) primer pair (FIG. 14). PCR analysis was performed under same PCR reaction condition as described in Example 4. PCR testing with T10/T13 on two transformants exhibited the expected size, 2883 bp, on 1% agarose gel. The result indicated the correct integration of the fused kanamycin selectable marker-P_{T5} promoter DNA fragment along with P. stewartii crtE gene into the inter-operon region located at 81.2 min of E. coli chromosome, yielding E. coli P_{T5}-crtE.

### Example 12

Chromosomal Integration of the P. stewartii crtIB Genes in E. coli P_{T5}-crtE to Construct P_{T5}-crtEIB—Preparation of Two Linear DNA Fragments: (1) Fused Kanamycin Selectable Marker-P_{T5} Promoter-P. stewartii crtE Gene and (2) P. stewartii crtI Genes, Electro-Transformation, and Elimination of the Antibiotic Selectable Marker

The linear DNA fragment containing the fused kanamycin selectable marker-P_{T5} promoter-P. stewartii crtE gene was synthesized by PCR from the genomic DNA of E. coli P_{T5}-crtE with primer pairs, T10 (SEQ ID NO:27) which contains a priming sequence (26 bp) corresponding to the 162 bases in the upstream region of the integration site of the fused kanamycin selectable marker-P_{T5} promoter-crtE gene in E. coli and B1 (crtIB) (5′-TCCTCCAGCATTAAGCCTGCCGTCGCCTTTTAACTGACGGCAGCG AGTTTTTTGTC-3′)(SEQ ID NO:28) which contains an h14 homology arm (underlined, 29 bp) chosen to match sequences in the downstream region of the crtI start codon and a priming sequence (27 bp) (FIG. 13). The linear DNA fragment containing P. stewartii crtIB gene was synthesized by PCR from pPCB15 (FIG. 5) with primer pairs, T2(crtIB) (5′-TTTGACAAAAAACTCGCTGCCGTCAGTTAAAAGGCGACGGCAGGCTT AATGCTG-3′)(SEQ ID NO:29) which contains an h14 homology arm (underlined, 30 bp) chosen to match a sequence in the 3′-end region of the fused kanamycin selectable marker-P_{T5} promoter-crtE gene and a priming sequence (24 bp) and B2(crtIB) (5′-AGAATGACCAGCTGGATGCATTATCTTTATTTGGATCATTGAGGGCTA GATCGGGCGCTGCCAGA-3′)(SEQ ID NO:30) which contains an h12 homology arm (underlined, 45 bp) chosen to match a sequence in the inter-operon region located at 81.2 min of the E. coli chromosome and a priming sequence (20 bp) (FIGS. 5 and 13). The underlined sequences illustrate each respective homology arm, while the remainder is the priming sequences for hybridization to complementary nucleotide sequences on the template DNA for the PCR reaction. The two resultant PCR fragments were the fused kanamycin selectable marker-P_{T5} promoter-P. stewartii crtE gene containing the homology region (162 bp) at the 5′-end and homology arm (h13), and the P. stewartii crtIB genes containing the homology arms (h14 and h12) as illustrated in FIG. 13.

The PCR reaction, purification and electro-transformation were performed as described in Example 2 except for omitting the step of transforming the host cell with the reporter plasmid, pPCB15, as described in Example 11. Both the fused kanamycin selectable marker-P_{T5} promoter-P. stewartii crtE gene PCR products (1-5 μg) and the P. stewartii crtIB PCR products (1-5 μg) were co-transformed into an E. coli host cell expressing the λ-Red recombinase system by electroporation as previously described in Example 2. Transformants were selected on 25 μg/mL kanamycin LB plates at 37° C. After incubating the plate at 37° C. overnight, one kan^{R} resistant transformant was selected. The kanamycin selectable marker was eliminated according to the method as described in Example 3.

### Example 13

Confirmation of Chromosomal Integration of the P. stewartii crtE and crtEIB Genes in E. coli P_{T5}-crtEIB

The selected kan^{R} resistant transformant was PCR analyzed with different combinations of specific primer pairs, T10 and T2, T2(T5) and T12 (5′-CTAGATCGGGCGCTGCCAGAGATGA-3′)(SEQ ID NO:32), T11(5′-ACACGTTCACCTTACTGGCATTTCG-3′)(SEQ ID NO:31) and T13, and T10 and T13 (FIG. 14). Test primers were chosen to amplify sequences located either in the vicinity of the integration region of the kanamycin selectable marker-P_{T5} promoter-crtE fragment or the crtIB genes (FIG. 14). PCR analysis was performed under same PCR reaction condition as described in Example 4. PCR test with T10 and T2, T2(T5 and T12, T11 and T13, and T10 and T13 exhibited the expected sizes, 676 bp, 3472 bp, 3478 bp and 5288 bp on 1% agarose gel, respectively. The elimination of the kanamycin selectable marker was confirmed by PCR fragment analysis (FIG. 14). PCR fragment analysis with primer pair T10 and T2 exhibited no product formation as expected. PCR analysis with primer pairs T2(T5) and T12, T11 and T13, and T10 and T13 exhibited the expected PCR product sizes of 3472 bp, 3478 bp, and 3895 bp on 1% agarose gel, respectively. The results indicated the correct integration of the fused kanamycin selectable marker-P_{T5} promoter-P. stewartii crtE gene DNA fragment and P. stewartii crtIB genes into the inter-operon region located at 81.2 min of E. coli chromosome, yielding E. coli P_{T5}-crtEIB.

The functional expression of the enzymes geranylgeranyl pyrophosphate synthase (CrtE), phytoene synthase (CrtB) and phytoene dehydrogenase (CrtI) in E. coli P_{T5}-crtEIB was confirmed by the synthesis of lycopene based on the production of pink pigment. After extracting lycopene with acetone as described in Example 5, lycopene production by E. coli P_{T5}-crtEIB was confirmed spectrophotometrically by observing λₘₐₓ peaks at 444, 470, and 502 nm, values characteristic of lycopene.

What is claimed is:

1. A method for the directed integration of an expressible DNA fragment lacking a selectable marker into a bacterial chromosome comprising:
a) providing at least one first recombination element having the general structure in the 5′ to 3′ direction:
5′-RR1-RS-SM-RS-RR2-3′; wherein
(i) RR1 is a first recombination region of about 10 to 50 bases;
(ii) RS is a recombination site responsive to a site-specific recombinase;
(iii) SM is a DNA fragment encoding a selectable marker; and
(iv) RR2 is a second recombination region of about 10 to 50 bases;
b) providing at least one second recombination element having the general structure in a 5′ to 3′ direction:
X-RR3; wherein
(i) X is an expressible DNA fragment having homology to the second recombination region; and
(ii) RR3 is a third recombination of about 10-50 bases;
c) providing a recombination proficient bacterial host harboring a λ-Red recombinase system, having a bacterial chromosome comprising:
(i) a first chromosomal region having homology to said first recombination region;
(ii) a second chromosomal region having homology to said third recombination region;
d) transforming said recombination proficient host with the first and second recombination elements, wherein both elements are integrated into the bacterial chromosome between the first and second chromosomal regions forming a construct having the general structure in the 5′ to 3′ direction;
5′-RR1-RS-SM-RS-RR2-X-RR3;
e) selecting and isolating transformed hosts having the construct of (d) on the basis of the selectable marker;
f) expressing a site-specific recombinase in the isolated hosts of (e) wherein the selectable marker is excised from the chromosome and whereby the expressible DNA fragment is inserted into the bacterial chromosome, lacking the selectable marker.

2. A method for the directed integration of an expressible DNA fragment lacking a selectable marker into a bacterial chromosome comprising:
a) providing at least one first recombination element having the general structure in the 5′ to 3′ direction:
5′-RR1-RS-SM-RS-Y-RR2-3′; wherein
(i) RR1 is a first recombination region of about 10 to 50 bases;
(ii) RS is a recombination site responsive to a site-specific recombinase;
(iii) SM is a DNA fragment encoding a selectable marker;
(iv) Y is a first expressible DNA fragment; and
(v) RR2 is a second recombination region of about 10 to 50 bases;
b) providing at least one second recombination element having the general structure in a 5′ to 3′ direction:
5′-X-RR3-3′; wherein
(i) X is a second expressible DNA fragment having homology to the second recombination region; and
(ii) RR3 is a third recombination of about 10-50 bases;
c) providing a recombination proficient bacterial host harboring a λ-Red recombinase system, and having a bacterial chromosome comprising:
(i) a first chromosomal region having homology to said first recombination region;
(ii) a second chromosomal region having homology to said third recombination region;
d) transforming said recombination proficient host with the first and second recombination elements, wherein both elements are integrated into the bacterial chromosome between the first and second chromosomal regions forming a construct having the general structure in the 5′ to 3′ direction;
5′-RR1-RS-SM-RS-Y-RR2-X-RR3;
e) selecting and isolating transformed hosts having the construct of (d) on the basis of the selectable marker;
f) expressing a site-specific recombinase in the isolated hosts of (e) wherein the selectable marker is excised from the chromosome and whereby the first and second expressible DNA fragments are inserted into the bacterial chromosome, lacking the selectable marker.

3. A method according to either one of claims 1 or 2 wherein either the first or second the expressible DNA fragment is selected from the group consisting of regulatory elements, promoters, genes, coding sequences, and open reading frames.

4. A method according to either one of claims 1 or 2 wherein the site-specific recombinase is expressed by a gene residing on a plasmid.

5. A method according to either one of claims 1 or 2 wherein said first chromosomal region is upstream of a bacterial promoter.

6. A method according to either one of claims 1 or 2 wherein said first chromosomal region is upstream of an inter-operon chromosomal integration site.

7. A method according to claim 3 wherein said expressible DNA fragment is a promoter selected from the group consisting of bacterial and phage promoters.

8. A method according to claim 7 wherein said promoter comprises positive and negative regulatory sites for control of a regulatory circuit.

9. A method according to claim 8 wherein said regulatory region comprises a lac operator site.

10. A method according to claim 7 wherein said promoter is selected from the group consisting of the phage T5 promoter, the phage T7 promoter, and the lac promoter.

11. A method according to either one of claims 1 or 2 wherein said selectable marker is selected from the group consisting of antibiotic resistance markers, enzymatic markers and amino acid biosynthesis enzymes.

12. A method according to either one of claims 1 or 2 wherein said recombination proficient host harboring a λ-Red recombinase system is selected from the group consisting of Escherichia, Salmonella, Acinetobacter, Methylomonas, Bacillus, and Pseudomonas.

13. A method according to either one of claims 1 or 2 wherein said recombination sites are selected from the group consisting of lox, frt, dif, and att.

14. A method according to claim 13 wherein said site-specific recombinase is selected from the group consisting of Cre, Flp, Xer, and Int.

15. A method according to either one of claims 1 or 2 wherein said recombination elements are generated by PCR.

16. A method according to either one of claims 1 or 2 wherein said recombination elements are from about 25 bases to about 4000 bases.

17. A method for the integration of a foreign promoter in place of a bacterial chromosomal promoter in a recombination proficient host cell comprising:
a) providing at least one first recombination element having the general structure in the 5′ to 3′ direction:
5′-RR1-RS-SM-RS-RR2-3′; wherein
(i) RR1 is a first recombination region of about 10 to 50 bases;
(ii) RS is a recombination site responsive to a site-specific recombinase;
(iii) SM is a DNA fragment encoding a selectable marker; and
(iv) RR2 is a second recombination region of about 10 to 50 bases;
b) providing at least one second recombination element having the general structure in a 5′ to 3′ direction:
5′-FP-RR3-3′; wherein
(i) FP is a promoter foreign to the recombination proficient host cell having homology to the second recombination region; and
(ii) RR3 is a third recombination of about 10-50 bases;
c) providing a recombination proficient bacterial host harboring a λ-Red recombinase system, having a bacterial chromosome comprising:
(i) a first chromosomal region upstream of a bacterial promoter having homology to said first recombination region;
(ii) a second chromosomal region, downstream of said bacterial promoter having homology to said third recombination region;
d) transforming said recombination proficient host with the first and second recombination elements, wherein both elements are integrated into the bacterial chromosome between the first and second chromosomal regions forming a construct having the general structure in the 5′ to 3′ direction;
5′-RR1-RS-SM-RS-RR2-FP-RR3;
e) selecting and isolating transformed hosts having the construct of (d) on the basis of the selectable marker;
f) expressing a site-specific recombinase in the isolated hosts of (e) wherein the selectable marker is excised from the chromosome and whereby the foreign promoter is inserted into the bacterial chromosome in place of the bacterial promoter.

18. A method for the integration of an unlinked foreign promoter and foreign open reading frame into a bacterial chromosome in a recombination proficient host cell comprising:
a) providing at least one first recombination element having the general structure in the 5′ to 3′ direction:
5′-RR1-RS-SM-RS-FP-RR2-3′; wherein
(i) RR1 is a first recombination region of about 10 to 50 bases;
(ii) RS is a recombination site responsive to a site-specific recombinase;
(iii) SM is a DNA fragment encoding a selectable marker;
(iv) FP is a promoter foreign to the recombination proficient host cell; and
(iv) RR2 is a second recombination region of about 10 to 50 bases;
b) providing at least one second recombination element having the general structure in a 5′ to 3′ direction:
5′-FO-RR3-3′; wherein
(i) FO is an open reading frame foreign to the recombination proficient host cell having homology to the second recombination region; and
(ii) RR3 is a third recombination of about 10-50 bases;
c) providing a recombination proficient bacterial host harboring a λ-Red recombinase system, having a bacterial chromosome comprising:
(i) a first chromosomal region upstream of a bacterial intra-operon chromosomal integration site having homology to said first recombination region;
(ii) a second chromosomal region, downstream of said bacterial intra-operon chromosomal integration site having homology to said third recombination region;
d) transforming said recombination proficient host with the first and second recombination elements, wherein both elements are integrated into the bacterial chromosome between the first and second chromosomal regions forming a construct having the general structure in the 5′ to 3′ direction;
5′-RR1-RS-SM-RS-FP-RR2-FO-RR3;
e) selecting and isolating transformed hosts having the construct of (d) on the basis of the selectable marker;
f) expressing a site-specific recombinase in the isolated hosts of (e) wherein the selectable marker is excised from the chromosome and whereby the foreign promoter and foreign open reading frame are inserted into the bacterial chromosome.

19. A method for the integration of a foreign gene comprising a regulatory region and foreign open reading frame into a bacterial chromosome in a recombination proficient host cell comprising:
a) providing at least one first recombination element having the general structure in the 5′ to 3′ direction:
5′-RR1-RS-SM-RS-FG-RR2-3′; wherein
(i) RR1 is a first recombination region of about 10 to 50 bases;
(ii) RS is a recombination site responsive to a site-specific recombinase;
(iii) SM is a DNA fragment encoding a selectable marker;
(iv) FG is a gene comprising a regulatory region, foreign to the recombination proficient host cell; and
(iv) RR2 is a second recombination region of about 10 to 50 bases;
b) providing at least one second recombination element having the general structure in a 5′ to 3′ direction:
5′-FO-RR3-3′; wherein
(i) FO is an open reading frame foreign to the recombination proficient host cell having homology to the second recombination region; and
(ii) RR3 is a third recombination of about 10-50 bases;
c) providing a recombination proficient bacterial host harboring a λ-Red recombinase system, having a bacterial chromosome comprising:
(i) a first chromosomal region upstream of a bacterial intra-operon chromosomal integration site having homology to said first recombination region;
(ii) a second chromosomal region, downstream of said bacterial intra-operon chromosomal integration site having homology to said third recombination region;
d) transforming said recombination proficient host with the first and second recombination elements, wherein both elements are integrated into the bacterial chromosome between the first and second chromosomal regions forming a construct having the general structure in the 5′ to 3′ direction;
5′-RR1-RS-SM-RS-FG-RR2-FO-RR3;
e) selecting and isolating transformed hosts having the construct of (d) on the basis of the selectable marker;
f) expressing a site-specific recombinase in the isolated hosts of (e) wherein the selectable marker is excised from the chromosome and whereby the foreign promoter and foreign open reading frame are inserted into the bacterial chromosome.

20. A method according to any one of claims 17-19 wherein the site-specific recombinase is expressed by a gene residing on a plasmid.

21. A method according to claim 17 wherein said promoter is selected from the group consisting of bacterial and phage promoters.

22. A method according to claim 21 wherein said promoter comprises positive and negative regulatory sites for control of regulatory circuit.

23. A method according to claim 22 wherein said regulatory region comprises a lac operator site.

24. A method according to claim 21 wherein said promoter is selected from the group consisting of the phage T5 promoter, the phage T7 promoter, and the lac promoter.

25. A method according to any one of claims 17-19 wherein said recombination proficient host harboring a λ-Red recombinase system is selected from the group consisting of Escherichia, Salmonella, Acinetobacter, Methylomonas, Bacillus, and Pseudomonas.

26. A method according to any one of claims 17-19 wherein said recombination sites are selected from the group consisting of lox, frt, dif, and att.

27. A method according to claim 26 wherein said site-specific recombinase is selected from the group consisting of Cre, Flp, Xer, and Int.

28. A method according to any one of claims 17-19 wherein said recombination elements are generated by PCR.

29. A method according to either one of claims 17-19 wherein said recombination elements are from about 25 bases to about 4000 bases.

30. A method according to any of claims 1, 2, 17-19 wherein steps (d)-(f) are repeated one or more times.


SEQUENCE LISTING (37 sequences)

SEQ ID NO: 1 - DNA, 61 bp, Artificial sequence
  Primer T1(dxs)
tggaagcgctagcggactacatcatccagcgtaataaataacgtcttgagcgattgtgtag

SEQ ID NO: 2 - DNA, 70 bp, Artificial sequence
  Primer B1(T5)
ctcgaggtgaagacgaaagggcctcgtgatacgcctatttttataggttacatatgaatatcctccttag

SEQ ID NO: 3 - DNA, 32 bp, Artificial sequence
  Primer T2(T5)
taacctataaaaataggcgtatcacgaggccc

SEQ ID NO: 4 - DNA, 70 bp, Artificial sequence
  Primer B2(dxs)
ggagtcgaccagtgccagggtcgggtatttggcaatatcaaaactcatagttaatttctcctctttaatg

SEQ ID NO: 5 - DNA, 26 bp, Artificial sequence
  Primer T1
cgccagtcgctgaaacaactggctga

SEQ ID NO: 6 - DNA, 20 bp, Artificial sequence
  Primer T2
cagtcatagccgaatagcct

SEQ ID NO: 7 - DNA, 20 bp, Artificial sequence
  Primer T3
cggtgccctgaatgaactgc

SEQ ID NO: 8 - DNA, 26 bp, Artificial sequence
  Primer T4
tggcaacagtcgtagctcctgggtgg

SEQ ID NO: 9 - DNA, 65 bp, Artificial sequence
  Primer T(idi)
tctgatgcgcaagctgaagaaaaatgagcatggagaataatatgacgtcttgagcgattgtgtag

SEQ ID NO: 10 - DNA, 68 bp, Artificial sequence
  Primer B(idi)
tgggaactccctgtgcattcaataaaatgacgtgttccgtttgcatagttaatttctcctctttaatg

SEQ ID NO: 11 - DNA, 25 bp, Artificial sequence
  Primer T5
tcatgctgacctggtgaaggaatcc

SEQ ID NO: 12 - DNA, 25 bp, Artificial sequence
  Primer T6
tcatgctgacctggtgaaggaatcc

SEQ ID NO: 13 - DNA, 42 bp, Artificial sequence
  Primer Tt5
atcatgacattaacatatgaaaataggcgtatcacgaggccc

SEQ ID NO: 14 - DNA, 38 bp, Artificial sequence
  Primer Bt5
gatgcgatcccatatgagttaatttctcctctttaatg

SEQ ID NO: 15 - DNA, 1860 bp, Methylomonas 16a
atgaaactgaccaccgactatcccttgcttaaaaacatccacacgccggcggacatacgcgcgctgtccaaggaccagctccagcaactggctgacgaggtgcgcggctatctgacccacacggtcagcatttccggcggccattttgcggccggcctcggcaccgtggaactgaccgtggccttgcattatgtgttcaatacccccgtcgatcagttggtctgggacgtgggccatcaggcctatccgcacaagattctgaccggtcgcaaggagcgcatgccgaccattcgcaccctgggcggggtgtcagcctttccggcgcgggacgagagcgaatacgatgccttcggcgtcggccattccagcacctcgatcagcgcggcactgggcatggccattgcgtcgcagctgcgcggcgaagacaagaagatggtagccatcatcggcgacggttccatcaccggcggcatggcctatgaggcgatgaatcatgccggcgatgtgaatgccaacctgctggtgatcttgaacgacaacgatatgtcgatctcgccgccggtcggggcgatgaacaattatctgaccaaggtgttgtcgagcaagttttattcgtcggtgcgggaagagagcaagaaagctctggccaagatgccgtcggtgtgggaactggcgcgcaagaccgaggaacacgtgaagggcatgatcgtgcccggtaccttgttcgaggaattgggcttcaattatttcggcccgatcgacggccatgatgtcgagatgctggtgtcgaccctggaaaatctgaaggatttgaccgggccggtattcctgcatgtggtgaccaagaagggcaaaggctatgcgccagccgagaaagacccgttggcctaccatggcgtgccggctttcgatccgaccaaggatttcctgcccaaggcggcgccgtcgccgcatccgacctataccgaggtgttcggccgctggctgtgcgacatggcggctcaagacgagcgcttgctgggcatcacgccggcgatgcgcgaaggctctggtttggtggaattctcacagaaatttccgaatcgctatttcgatgtcgccatcgccgagcagcatgcggtgaccttggccgccggccaggcctgccagggcgccaagccggtggtggcgatttattccaccttcctgcaacgcggttacgatcagttgatccacgacgtggccttgcagaacttagatatgctctttgcactggatcgtgccggcttggtcggcccggatggaccgacccatgctggcgcctttgattacagctacatgcgctgtattccgaacatgctgatcatggctccagccgacgagaacgagtgcaggcagatgctgaccaccggcttccaacaccatggcccggcttcggtgcgctatccgcgcggcaaagggcccggggcggcaatcgatccgaccctgaccgcgctggagatcggcaaggccgaagtcagacaccacggcagccgcatcgccattctggcctggggcagcatggtcacgcctgccgtcgaagccggcaagcagctgggcgcgacggtggtgaacatgcgtttcgtcaagccgttcgatcaagccttggtgctggaattggccaggacgcacgatgtgttcgtcaccgtcgaggaaaacgtcatcgccggcggcgctggcagtgcgatcaacaccttcctgcaggcgcagaaggtgctgatgccggtctgcaacatcggcctgcccgaccgcttcgtcgagcaaggtagtcgcgaggaattgctcagcctggtcggcctcgacagcaagggcatcctcgccaccatcgaacagttttgcgct

SEQ ID NO: 16 - DNA, 65 bp, Artificial sequence
  Primer T1(dxs16a)
cactaacgcccgcacattgctgcgggctttttgattcatttcgcacgtcttgagcgattgtgtag

SEQ ID NO: 17 - DNA, 51 bp, Artificial sequence
  Primer B1(dxs16a)
agtagagggaagtctttggaaagagccatagttaatttctcctctttaatg

SEQ ID NO: 18 - DNA, 55 bp, Artificial sequence
  Primer T2(dxs16a)
acagaattcattaaagaggagaaattaactatggctctttccaaagacttccctc

SEQ ID NO: 19 - DNA, 68 bp, Artificial sequence
  Primer B2(dxs16a)
aggagcgaagtgattatcagtatgctgttcatatagcctcgaattatcaagcgcaaaactgttcgatg

SEQ ID NO: 20 - DNA, 25 bp, Artificial sequence
  Primer T7
accggatatcaccacttatctgctc

SEQ ID NO: 21 - DNA, 25 bp, Artificial sequence
  Primer T8
atgctgaccgtgtgggtcagatagc

SEQ ID NO: 22 - DNA, 25 bp, Artificial sequence
  Primer T9
gcgatattgtatgtctgattcagga

SEQ ID NO: 23 - DNA, 65 bp, Artificial sequence
  Primer T1(crtE)
agccgtcgcaggaggaacaactcatatcatcattgcgatctcgaccgtcttgagcgattgtgtag

SEQ ID NO: 24 - DNA, 51 bp, Artificial sequence
  Primer B1(crtE)
tgaacgtgtttttttgcgcagaccgtcatagttaatttctcctctttaatg

SEQ ID NO: 25 - DNA, 55 bp, Artificial sequence
  Primer T2(crtE)
acagaattcattaaagaggagaaattaactatgacggtctgcgcaaaaaaacacg

SEQ ID NO: 26 - DNA, 65 bp, Artificial sequence
  Primer B2(crtE)
agaatgaccagctggatgcattatctttatttggatcattgagggttaactgacggcagcgagtt

SEQ ID NO: 27 - DNA, 26 bp, Artificial sequence
  Primer T10
ccatgaccctacattgtgatctatag

SEQ ID NO: 28 - DNA, 56 bp, Artificial sequence
  Primer B1(crtIB)
tcctccagcattaagcctgccgtcgccttttaactgacggcagcgagttttttgtc

SEQ ID NO: 29 - DNA, 54 bp, Artificial sequence
  Primer T2(crtIB)
tttgacaaaaaactcgctgccgtcagttaaaaggcgacggcaggcttaatgctg

SEQ ID NO: 30 - DNA, 65 bp, Artificial sequence
  Primer B2(crtIB)
agaatgaccagctggatgcattatctttatttggatcattgagggctagatcgggcgctgccaga

SEQ ID NO: 31 - DNA, 25 bp, Artificial sequence
  Primer T11
acacgttcaccttactggcatttcg

SEQ ID NO: 32 - DNA, 25 bp, Artificial sequence
  Primer T12
ctagatcgggcgctgccagagatga

SEQ ID NO: 33 - DNA, 25 bp, Artificial sequence
  Primer T13
ggaaccattgaactggaccctaacg

SEQ ID NO: 34 - DNA, 3267 bp, Artificial sequence
  Plasmid pKD4
agattgcagcattacacgtcttgagcgattgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatggaccatggctaattcccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgcggccgcaaaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcatcgatggccccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgc

SEQ ID NO: 35 - DNA, 6329 bp, Artificial sequence
  Plasmid pKD46
catcgatttattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 36 - DNA, 3423 bp, Artificial sequence
  Plasmid pSUH5
agattgcagcattacacgtcttgagcgattgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcactataaaaataggcgtatcacgaggccctttcgtcttcacctcgagaaatcataaaaaatttatttgctttgtgagcggataacaattataatagattcaattgtgagcggataacaatttcacacagaattcattaaagaggagaaattaactcatatggaccatggctaattcccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgcggccgcaaaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcatcgatggccccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgc

SEQ ID NO: 37 - DNA, 8609 bp, Artificial sequence
  Plasmid pPCB15
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatatacactccgctagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccaaacgaattcgcccttttgacggtctgcgcaaaaaaacacgttcaccttactggcatttcggctgagcagttgctggctgatatcgatagccgccttgatcagttactgccggttcagggtgagcgggattgtgtgggtgccgcgatgcgtgaaggcacgctggcaccgggcaaacgtattcgtccgatgctgctgttattaacagcgcgcgatcttggctgtgcgatcagtcacgggggattactggatttagcctgcgcggttgaaatggtgcatgctgcctcgctgattctggatgatatgccctgcatggacgatgcgcagatgcgtcgggggcgtcccaccattcacacgcagtacggtgaacatgtggcgattctggcggcggtcgctttactcagcaaagcgtttggggtgattgccgaggctgaaggtctgacgccgatagccaaaactcgcgcggtgtcggagctgtccactgcgattggcatgcagggtctggttcagggccagtttaaggacctctcggaaggcgataaaccccgcagcgccgatgccatactgctaaccaatcagtttaaaaccagcacgctgttttgcgcgtcaacgcaaatggcgtccattgcggccaacgcgtcctgcgaagcgcgtgagaacctgcatcgtttctcgctcgatctcggccaggcctttcagttgcttgacgatcttaccgatggcatgaccgataccggcaaagacatcaatcaggatgcaggtaaatcaacgctggtcaatttattaggctcaggcgcggtcgaagaacgcctgcgacagcatttgcgcctggccagtgaacacctttccgcggcatgccaaaacggccattccaccacccaactttttattcaggcctggtttgacaaaaaactcgctgccgtcagttaaggatgctgcatgagccattttgcggtgatcgcaccgccctttttcagccatgttcgcgctctgcaaaaccttgctcaggaattagtggcccgcggtcatcgtgttacgttttttcagcaacatgactgcaaagcgctggtaacgggcagcgatatcggattccagaccgtcggactgcaaacgcatcctcccggttccttatcgcacctgctgcacctggccgcgcacccactcggaccctcgatgttacgactgatcaatgaaatggcacgtaccagcgatatgctttgccgggaactgcccgccgcttttcatgcgttgcagatagagggcgtgatcgttgatcaaatggagccggcaggtgcagtagtcgcagaagcgtcaggtctgccgtttgtttcggtggcctgcgcgctgccgctcaaccgcgaaccgggtttgcctctggcggtgatgcctttcgagtacggcaccagcgatgcggctcgggaacgctataccaccagcgaaaaaatttatgactggctgatgcgacgtcacgatcgtgtgatcgcgcatcatgcatgcagaatgggtttagccccgcgtgaaaaactgcatcattgtttttctccactggcacaaatcagccagttgatccccgaactggattttccccgcaaagcgctgccagactgctttcatgcggttggaccgttacggcaaccccaggggacgccggggtcatcaacttcttattttccgtccccggacaaaccccgtatttttgcctcgctgggcaccctgcagggacatcgttatggcctgttcaggaccatcgccaaagcctgcgaagaggtggatgcgcagttactgttggcacactgtggcggcctctcagccacgcaggcaggtgaactggcccggggcggggacattcaggttgtggattttgccgatcaatccgcagcactttcacaggcacagttgacaatcacacatggtgggatgaatacggtactggacgctattgcttcccgcacaccgctactggcgctgccgctggcatttgatcaacctggcgtggcatcacgaattgtttatcatggcatcggcaagcgtgcgtctcggtttactaccagccatgcgctggcgcggcagattcgatcgctgctgactaacaccgattacccgcagcgtatgacaaaaattcaggccgcattgcgtctggcaggcggcacaccagccgccgccgatattgttgaacaggcgatgcggacctgtcagccagtactcagtgggcaggattatgcaaccgcactatgatctcattctggtcggtgccggtctggctaatggccttatcgcgctccggcttcagcaacagcatccggatatgcggatcttgcttattgaggcgggtcctgaggcgggagggaaccatacctggtcctttcacgaagaggatttaacgctgaatcagcatcgctggatagcgccgcttgtggtccatcactggcccgactaccaggttcgtttcccccaacgccgtcgccatgtgaacagtggctactactgcgtgacctcccggcatttcgccgggatactccggcaacagtttggacaacatttatggctgcataccgcggtttcagccgttcatgctgaatcggtccagttagcggatggccggattattcatgccagtacagtgatcgacggacggggttacacgcctgattctgcactacgcgtaggattccaggcatttatcggtcaggagtggcaactgagcgcgccgcatggtttatcgtcaccgattatcatggatgcgacggtcgatcagcaaaatggctaccgctttgtttataccctgccgctttccgcaaccgcactgctgatcgaagacacacactacattgacaaggctaatcttcaggccgaacgggcgcgtcagaacattcgcgattatgctgcgcgacagggttggccgttacagacgttgctgcgggaagaacagggtgcattgcccattacgttaacgggcgataatcgtcagttttggcaacagcaaccgcaagcctgtagcggattacgcgccgggctgtttcatccgacaaccggctactccctaccgctcgcggtggcgctggccgatcgtctcagcgcgctggatgtgtttacctcttcctctgttcaccagacgattgctcactttgcccagcaacgttggcagcaacaggggtttttccgcatgctgaatcgcatgttgtttttagccggaccggccgagtcacgctggcgtgtgatgcagcgtttctatggcttacccgaggatttgattgcccgcttttatgcgggaaaactcaccgtgaccgatcggctacgcattctgagcggcaagccgcccgttcccgttttcgcggcattgcaggcaattatgacgactcatcgttgaagagcgactacatgaaaccaactacggtaattggtgcgggctttggtggcctggcactggcaattcgtttacaggccgcaggtattcctgttttgctgcttgagcagcgcgacaagccgggtggccgggcttatgtttatcaggagcagggctttacttttgatgcaggccctaccgttatcaccgatcccagcgcgattgaagaactgtttgctctggccggtaaacagcttaaggattacgtcgagctgttgccggtcacgccgttttatcgcctgtgctgggagtccggcaaggtcttcaattacgataacgaccaggcccagttagaagcgcagatacagcagtttaatccgcgcgatgttgcgggttatcgagcgttccttgactattcgcgtgccgtattcaatgagggctatctgaagctcggcactgtgccttttttatcgttcaaagacatgcttcgggccgcgccccagttggcaaagctgcaggcatggcgcagcgtttacagtaaagttgccggctacattgaggatgagcatcttcggcaggcgttttcttttcactcgctcttagtgggggggaatccgtttgcaacctcgtccatttatacgctgattcacgcgttagaacgggaatggggcgtctggtttccacgcggtggaaccggtgcgctggtcaatggcatgatcaagctgtttcaggatctgggcggcgaagtcgtgcttaacgcccgggtcagtcatatggaaaccgttggggacaagattcaggccgtgcagttggaagacggcagacggtttgaaacctgcgcggtggcgtcgaacgctgatgttgtacatacctatcgcgatctgctgtctcagcatcccgcagccgctaagcaggcgaaaaaactgcaatccaagcgtatgagtaactcactgtttgtactctattttggtctcaaccatcatcacgatcaactcgcccatcataccgtctgttttgggccacgctaccgtgaactgattcacgaaatttttaaccatgatggtctggctgaggatttttcgctttatttacacgcaccttgtgtcacggatccgtcactggcaccggaagggtgcggcagctattatgtgctggcgcctgttccacacttaggcacggcgaacctcgactgggcggtagaaggaccccgactgcgcgatcgtatttttgactaccttgagcaacattacatgcctggcttgcgaagccagttggtgacgcaccgtatgtttacgccgttcgatttccgcgacgagctcaatgcctggcaaggttcggccttctcggttgaacctattctgacccagagcgcctggttccgaccacataaccgcgataagcacattgataatctttatctggttggcgcaggcacccatcctggcgcgggcattcccggcgtaatcggctcggcgaaggcgacggcaggcttaatgctggaggacctgatttgacgaatacgtcattactgaatcatgccgtcgaaaccatggcggttggctcgaaaagctttgcgactgcatcgacgcttttcgacgccaaaacccgtcgcagcgtgctgatgctttacgcatggtgccgccactgcgacgacgtcattgacgatcaaacactgggctttcatgccgaccagccctcttcgcagatgcctgagcagcgcctgcagcagcttgaaatgaaaacgcgtcaggcctacgccggttcgcaaatgcacgagcccgcttttgccgcgtttcaggaggtcgcgatggcgcatgatatcgctcccgcctacgcgttcgaccatctggaaggttttgccatggatgtgcgcgaaacgcgctacctgacactggacgatacgctgcgttattgctatcacgtcgccggtgttgtgggcctgatgatggcgcaaattatgggcgttcgcgataacgccacgctcgatcgcgcctgcgatctcgggctggctttccagttgaccaacattgcgcgtgatattgtcgacgatgctcaggtgggccgctgttatctgcctgaaagctggctggaagaggaaggactgacgaaagcgaattatgctgcgccagaaaaccggcaggccttaagccgtatcgccgggcgactggtacgggaagcggaaccctattacgtatcatcaatggccggtctggcacaattacccttacgctcggcctgggccatcgcgacagcgaagcaggtgtaccgtaaaattggcgtgaaagttgaacaggccggtaagcaggcctgggatcatcgccagtccacgtccaccgccgaaaaattaacgcttttgctgacggcatccggtcaggcagttacttcccggatgaagacgtatccaccccgtcctgctcatctctggcagcgcccgatctagccgcatgcctttctctcagcgtcgcctgaagtttagataacggtggcgcgtacagaaaaccaaaggacacgcagccctcttttccccttacagcatgatgcatacggtgggccatgtataaccgtttcaggtagcctttgcgcggtatgtagcggaacggccagcgctggtgtaccagtccgtcgtggaccataaaatacagtaaaccataagcggtcatgcctgcaccaatccactggagcggccagattcctgtactgccgaagtaaatcagggcaatcgacacaatggcgaataccacggcatagagatcgttaacttcaaatgcgcctttacgcggttcatgatgtgaaagatgccagccccaaccccagccgtgcatgatgtatttatgtgccagtgcagcaaccacttccatgccgaccacggtgacaaacacgatcagggcattccaaatccacaacataatttctcaagggcgaattcgcggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattt